(19) United States
(12) Patent Application Publication
(10) Pub. No.: US 2006/0069087 A1
(43) Pub. Date: Mar. 30, 2006

(54) Histamine-3 receptor antagonists

(75) Inventor: Travis T. Wager, New London, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/224,913

(22) Filed: Sep. 12, 2005

Related U.S. Application Data
(60) Provisional application No. 60/613,796, filed on Sep. 27, 2004.

(51) Int. Cl. A61K 31/5513; A61K 31/541; A61K 31/5377; A61K 31/496; A61K 31/4178; A61K 31/405; A61K 31/454; C07D 403/14

(52) U.S. Cl. 514/221; 514/323; 514/397; 514/414; 514/232.5; 514/227.8; 514/254.09; 540/568; 544/60; 544/373; 546/201; 548/465; 544/143; 548/312.1

(57) ABSTRACT

This invention is directed to a compound of the formula I 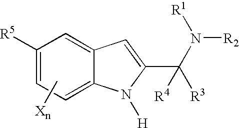 as defined herein, or a pharmaceutically acceptable salt thereof; a pharmaceutical composition containing a compound of formula I, a method of treatment of a disorder or condition that may be treated by antagonizing histamine H3 receptors, the method comprising administering to a mammal in need of such treatment a compound of formula I as described above, and a method of treatment of a disorder or condition selected from the group consisting of depression, mood disorders, schizophrenia, anxiety disorders, Alzheimer's disease, attention-deficit disorder (ADD), attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula I as described above.

### BACKGROUND OF THE INVENTION

This invention is directed to compounds of formula I described herein, to a pharmaceutical composition comprising such compounds, and to methods of treatment of disorders or conditions that may be treated by antagonizing histamine-3 (H3) receptors using such compounds. The histamine-3 (H3) receptor antagonists of the invention are useful for treating anxiety disorders, including, for example, generalized anxiety disorder, panic disorder, PTSD, and social anxiety disorder; mood adjustment disorders, including depressed mood, mixed anxiety and depressed mood, disturbance of conduct, and mixed disturbance of conduct and depressed mood; age-associated learning and mental disorders, including Alzheimer's disease; attention adjustment disorders, such as attention-deficit disorders, or other cognitive disorders due to general medical conditions; attention-deficit hyperactivity disorder; psychotic disorders including schizoaffective disorders and schizophrenia; sleep disorders, including narcolepsy and enuresis; obesity; dizziness, epilepsy, and motion sickness. The H3 receptor antagonists of the invention are also useful for treating, for example, allergy, allergy-induced airway (e.g., upper airway) responses, congestion (e.g., nasal congestion), hypotension, cardiovascular disease, diseases of the GI tract, hyper and hypo motility and acidic secretion of the gastrointestinal tract, sleeping disorders (e.g., hypersomnia, somnolence, and narcolepsy), disturbances of the central nervous system, attention deficit hyperactivity disorder (ADHD), hypo and hyperactivity of the central nervous system (for example, agitation and depression), and other CNS disorders (such as schizophrenia and migraine).

Histamine is a well-known mediator in hypersensitive reactions (e.g. allergies, hay fever, and asthma) that are commonly treated with antagonists of histamine or “antihistamines.” It has also been established that histamine receptors exist in at least two distinct types, referred to as H1 and H2 receptors.

A third histamine receptor (H3 receptor) is believed to play a role in neurotransmission in the central nervous system, where the H3 receptor is thought to be disposed presynaptically on histaminergic nerve endings (Nature, 302, S32-837 (1983)). The existence of the H3 receptor has been confirmed by the development of selective H3 receptor agonists and antagonists (Nature, 327, 117-123 (1987)) and has subsequently been shown to regulate the release of the neurotransmitters in both the central nervous system and peripheral organs, particularly the lungs, cardiovascular system and gastrointestinal tract.

A number of diseases or conditions may be treated with histamine-3 receptor ligands wherein the H3 ligand may be an antagonist, agonist or partial agonist, see: (Imamura et al., Circ. Res., (1996) 78, 475-481); (Imamura et. al., Circ. Res., (1996) 78, 863-869); (Lin et al., Brain Res. (1990) 523, 325-330); (Monti et al., Neuropsychopharmacology (1996) 15, 31 35); (Sakai, et al., Life Sci. (1991) 48, 2397-2404); (Mazurkiewiez-Kwilecki and Nsonwah, Can. J. Physiol. Pharmacol. (1989) 67, 75-78); (Panula, P. et al.,

Neuroscience (1998) 44, 465-481);. (Wada et al., Trends in Neuroscience (1991) 14,415); (Monti et al., Eur. J. Pharmacol. (1991) 205, 283); (Mazurkiewicz-Kwilecki and Nsonwah, Can. J. Physiol. Pharmacol. (1989) 67, 75-78); (Haas et al., Behav. Brain Res. (1995) 66, 41-44); (De Almeida and Izquierdo, Arch. Int. Pharmacodyn. (1986) 283, 193-198); (Kamei et al., Psychopharmacology (1990) 102, 312-318); (Kamei and Sakata, Japan. J. Pharmacol. (199 1) 57, 437-482); (Schwartz et al., Psychopharmacology; The fourth Generation of Progress, Bloom and Kupfer (eds.), Raven Press, New York, (1995) 3 97); (Shaywitz et al., Psychopharmacology (1984) 82, 73-77); (Dumery and Blozovski, Exp. Brain Res. (1987) 67, 61-69); (Tedford et al., J. Pharmacol. Exp. Ther. (1995) 275, 598-604); (Tedford et al., Soc. Neurosci. Abstr. (1996) 22, 22); (Yokoyama et al., Eur. J. Pharmacol. (1993) 234, 129); (Yokoyama and Iinuma, CNS Drugs (1996) 5, 321); (Onodera et al., Prog. Neurobiol. (1994) 42, 685); (Leurs and Timmerman, Prog. Drug Res. (1992) 39, 127); (The Histamine H3 Receptor, Leurs and Timmerman (ed.), Elsevier Science, Amsterdam, The Netherlands (1998); (Leurs et al., Trends in Pharm. Sci. (1998) 19, 177-183); (Phillips et al., Annual Reports in Medicinal Chemistry (1998) 33, 31-40); (Matsubara et al., Eur. J. Pharmacol. (1992) 224, 145); (Rouleau et al., J. Pharmacol. Exp. Ther. (1997) 281, 1085); (Adam Szelag, “Role of histamine H3-receptors in the proliferation of neoplastic cells in vitro”, Med. Sci. Monit., 4(5): 747-755, (1998)); (Fitzsimons, C., H. Duran, F. Labombarda, B. Molinari and E. Rivera, “Histamine receptors signalling in epidermal tumor cell lines with H-ras gene alterations”, Inflammation Res., 47 (Suppl. 1): S50-S51, (1998)); (R. Leurs, R. C. Vollinga and H. Timmerman, “The medicinal chemistry and therapeutic potentials of ligand of the histamine H3 receptor”, Progress in Drug Research 45: 170-165, (1995)); (R. Levi and N. C. E. Smith, “Histamine H3-receptors: A new frontier in myocardial ischemia”, J. Pharm. Exp. Ther., 292: 825-830, (2000)); (Hatta, E., K Yasuda and R. Levi, “Activation of histamine H3 receptors inhibits carrier-mediated norepinephrine release in a human model of protracted myocardial ischemia”, J. Pharm. Exp. Ther., 283: 494-500, (1997); (H. Yokoyama and K. Iinuma, “Histamine and Seizures: Implications for the treatment of epilepsy”, CNS Drugs, 5(5); 321-330, (1995)); (K. Hurukami, H. Yokoyama, K. Onodera, K. Iinuma and T. Watanabe, AQ-0 145, “A newly developed histamine H3 antagonist, decreased seizure susceptibility of electrically induced convulsions in mice”, Meth. Find. Exp. Clin. Pharmacol., 17(C): 70-73, (1995); (Delaunois A., Gustin P., Garbarg M., and Ansay M., “Modulation of acetylcholine, capsaicin and substance P effects by histamine H3 receptors in isolated perfused rabbit lungs”, European Journal of Pharmacology 277(2-3):243-50, (1995)); and (Dimitriadou, et al., “Functional relationship between mast cells and C-sensitive nerve fibres evidenced by histamine H3-receptor modulation in rat lung and spleen”, Clinical Science 87(2):151-63, (1994). Such diseases or conditions include cardiovascular disorders such as acute myocardial infarction; memory processes, dementia and cognition disorders such as Alzheimer's disease and attention-deficit hyperactivity disorder; neurological disorders such as Parkinson's disease, schizophrenia, depression, epilepsy, and seizures or convulsions; cancer such as cutaneous carcinoma,” medullary thyroid carcinoma and melanoma; respiratory disorders such as asthma; sleep disorders such as narcolepsy; vestibular dysfunction such as Meniere's disease; gastrointestinal disorders, inflammation, migraine, motion sickness, obesity, pain, and septic shock.

H3 receptor antagonists have also been previously described in, for example, WO 03/050099, WO 02/0769252, and WO 02/12224. The histamine H3 receptor (H3R) regulates the release of histamine and other neurotransmitters, including serotonin and acetylcholine. H3R is relatively neuron specific and inhibits the release of certain monoamines such as histamine. Selective antagonism of H3R raises brain histamine levels and inhibits such activities as food consumption while minimizing non-specific peripheral consequences. Antagonists of the receptor increase synthesis and release of cerebral histamine and other monoamines. By this mechanism, they induce a prolonged wakefulness, improved cognitive function, reduction in food intake and normalization of vestibular reflexes. Accordingly, the receptor is an important target for new therapeutics in Alzheimer disease, mood and attention adjustments, including attention deficit hyperactive disorder (ADHD), cognitive deficiencies, obesity, dizziness, schizophrenia, epilepsy, sleeping disorders, narcolepsy and motion sickness, and various forms of anxiety.

The majority of histamine H3 receptor antagonists to date resemble histamine in possessing an imidazole ring that may be substituted, as described, for example, in WO96/38142. Non-imidazole neuroactive compounds such as beta histamines (Arrang, Eur. J. Pharm. 1985, 111:72-84) demonstrated some histamine H3 receptor activity but with poor potency. EP 978512 and EP 0982300A2 disclose non-imidazole alkyamines as histamine H3 receptor antagonists. WO 02/12224 (Ortho McNeil Pharmaceuticals) describes non-imidazole bicyclic derivatives as histamine H3 receptor ligands. Other receptor antagonists have been described in WO02/32893 and WO02/06233.

This invention is directed to histamine-3 (H3) receptor antagonists of the invention useful for treating the conditions listed in the preceding paragraphs. The compounds of this invention are highly selective for the H3 receptor (vs. other histamine receptors), and possess remarkable drug disposition properties (pharmacokinetics). In particular, the compounds of this invention selectively distinguish H3R from the other receptor subtypes H1R, H2R. In view of the increased level of interest in histamine H3 receptor agonists, inverse agonists and antagonists in the art, novel compounds that interact with the histamine H3 receptor would be a highly desirable contribution to the art. The present invention provides such a contribution to the art being based on the finding that a novel class of biaryl amines has a high and specific affinity to the histamine H3 receptor.

### SUMMARY OF THE INVENTION

This invention is directed to a compound of the formula I 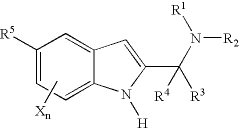
or a pharmaceutically acceptable salt thereof, wherein:

n=1, 2, or 3

Xₙ are independently selected from H, F, Cl, Br, I, C₁-C₆ alkyl (optionally substituted by F), C₁-C₆ alkoxyl (optionally substituted by F), (C₁-C₆ alkyl)-S(O)ₚ (optionally substituted by F, NO₂, COOH, COOR⁹, CONR¹⁰OR¹¹;

wherein R⁹ is hydrogen, C₁-C₆ alkyl (optionally substituted by F), aryl, heteroaryl, C₁-C₆ alkyl-aryl, C₁-C₆ alkyl-heteroaryl;

R¹⁰ and R¹¹ are chosen from the group consisting of hydrogen, C₁-C₆ alkyl, aryl, heteroaryl, C₁-C₆ alkyl-(aryl), or R¹⁰ and R¹¹ taken together with the nitrogen to which they are attached form a ring of 4-8 atoms with up to 3 additional heteroatoms including N, O, S; and

p=0, 1 or 2.

R¹ and R² are independently selected from the group consisting of hydrogen;

C₁-C₈ alkyl optionally substituted with 1 to 4 halogens or OH;
C₃-C₇ cycloalkyl;
3-8-membered heterocycloalkyl optionally substituted with a C₁-C₄ alkyl-carbonyl group;
C₆-C₁₀ arylsulfonyl optionally substituted with C₁-C₂ alkyl; and
5-10-membered heteroaryl;

R³ is selected from the group consisting of

C₁-C₈ alkyl optionally substituted with 1 to 4 halogens;
C₃-C₇ cycloalkyl;
C₆-C₁₄ aryl; or

R¹ and R² together with the nitrogen of the NR¹R² group form a 4-7 member ring, wherein one of the carbons in the ring is optionally replaced by O, S, NR⁶, or CO, and the ring is optionally fused to a C₆-C₁₀ arylene and is optionally substituted at a ring carbon with one or two C₁-C₄ alkyl groups, wherein R⁶ is

hydrogen;

C₁-C₈ alkyl optionally substituted with 1 to 4 halogens;

5-10-membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, cyano;

C₆-C₁₀ aryl optionally substituted with one or two C₁-C₂ alkyl; or

C₁-C₄ alkyl-carbonyl; or

R¹ and R³ together with the nitrogen of the NR¹R³ group form a 4-7 member ring, wherein one of the carbons in the ring is optionally replaced by O, S, NR^{6′}, or CO, and the ring is optionally fused to a C₆-C₁₀ arylene and is optionally substituted at a ring carbon with one or two C₁-C₄ alkyl groups, wherein R^{6′}is

hydrogen;

C₁-C₈ alkyl optionally substituted with 1 to 4 halogens;

5-10-membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, cyano;

C₆-C₁₀ aryl optionally substituted with one or two C₁-C₂ alkyl; or

C₁-C₄ alkyl-carbonyl;

R⁴ is

hydrogen, or

C₁-C₈ alkyl optionally substituted with 1 to 4 halogens;

R⁵ is —CHR^{7′}NR^{2″}R^{3″};

R^{2″} is hydrogen, C₁-C₈ alkyl optionally substituted with 1 to 4 halogens, C₃-C₇ cycloalkyl-C₀-C₄ alkyl, C₆-C₁₄ aryl-C₀-C₄ alkyl, 5-10-membered heteroaryl-C₀-C₄ alkyl, or C₆-C₁₄ aryl-C₀-C₄ alkylene-O—C₀-C₄ alkyl, wherein each C₀-C₄ alkyl and each C₀-C₄ alkylene is optionally substituted with one to four C₁-C₄ alkyl;

R^{3″} is hydrogen, C₁-C₈ alkyl optionally substituted with 1 to 4 halogens, C₆-C₁₄ aryl, C₆-C₁₄ arylcarbonyl-C₆-C₁₄ aryl, C₆-C₁₄ arylcarbonyl-3-8-membered heterocycloalkyl, C₃-C₈ cycloalkylcarbonyl-C₆-C₁₄ aryl, C₃-C₈ cycloalkylcarbonyl-3-8-membered heterocycloalkyl, 3-8-membered heterocycloalkyl, 3-8-membered heterocycloalkylcarbonyl-C₆-C₁₄ aryl, or 3-8-membered heterocycloalkylcarbonyl-3-8-membered heterocycloalkyl;

or R^{3″} and R^{2″} together with the nitrogen of the CHR^{7′}NR^{2″}R^{3″} group form a second 5-, 6- or 7-membered aliphatic ring, wherein one of the carbons in the second 5-, 6-, or 7-membered aliphatic ring is optionally replaced by O, S, NR¹¹, or C═O and the second 5-, 6-, or 7-membered aliphatic ring is optionally substituted with one or two C₁-C₄ alkyl, wherein R¹¹ is hydrogen, C₁-C₈ alkyl optionally substituted with 1 to 4 halogens, C₃-C₇ cycloalkyl-C₀-C₄ alkyl, C₆-C₁₄ aryl-C₀-C₄ alkyl, 5-10-membered heteroaryl-C₀-C₄ alkyl, or C₆-C₁₄ aryl-C₀-C₄ alkylene-O—C₀-C₄ alkyl, wherein each C₀-C₄ alkyl and each C₀-C₄ alkylene is optionally substituted with one to four C₁-C₄ alkyl; and

R^{7′} is hydrogen, C₁-C₈ alkyl optionally substituted with 1 to 4 halogens, C₃-C₇ cycloalkyl-C₀-C₄ alkyl, C₆-C₁₄ aryl-C₀-C₄ alkyl, 5-10-membered heteroaryl-C₀-C₄ alkyl, C₆-C₁₄ aryl-C₀-C₄ alkylene-O—C₀-C₄ alkyl, wherein each C₀-C₄ alkyl and each C₀-C₄ alkylene is optionally substituted with one to four C₁-C₄ alkyl, or SO₂C₁-C₁₀ alkyl.

The term “alkyl” refers to straight or branched chains of carbon atoms. Exemplary alkyl groups are C₁-C₆ alkyl groups which include methyl, ethyl, propyl, isopropyl, butyl, isobutyl, pentyl, isopentyl, hexyl, and the like, including all regioisomeric forms thereof, and straight and branched chain forms thereof. The term “alkyl” is also used to denote straight or branched chains of carbon atoms having one or more carbon-carbon double bonds, such as vinyl, allyl, butenyl, and the like, as well as straight or branched chains of carbon atoms having one or more carbon-carbon triple bonds, such as ethynyl, propargyl, butynyl, and the like. The term “aryl” denotes a cyclic, aromatic hydrocarbon. Examples of aryl groups include phenyl, naphthyl, anthracenyl, phenanthrenyl, and the like. The terms “alkoxy” and “aryloxy” denote “O-alkyl” and “O-aryl”, respectively. The term “cycloalkyl” denotes a cyclic group of carbon atoms, where the ring formed by the carbon atoms may be saturated or may comprise one or more carbon-carbon double bonds in the ring. Examples of cycloalkyl groups include cyclopropyl, cyclobutyl, cyclopentyl, cyclohexyl, cycloheptyl, and the like, as well as cyclopentenyl, cyclopentadienyl, cyclohexenyl, cyclohexadienyl, cyclobutadienyl, and the like. As used herein, the term “cycloalkyl” is also intended to denote a cyclic group comprising at least two fused rings, such as adamantanyl, decahydronaphthalinyl, norbornanyl, where the cyclic group may also have one or more carbon-carbon double bonds in one or both rings, such as in bicyclo[4.3.0]nona-3,6(1)-dienyl, dicyclopentadienyl, 1,2,3,4-tetrahydronaphthalinyl (tetralinyl), indenyl, and the like. The term “halogen” represents chloro, fluoro, bromo, and iodo. The term “heteroaryl” denotes a monocyclic or bicyclic aromatic group wherein one or more carbon atoms are replaced with heteroatoms selected from the group consisting of nitrogen, oxygen, and sulfur. If the heteroaryl group contains more than one heteroatom, the heteroatoms may be the same or different. Preferred heteroaryl groups are five- and six-member rings that contain from one to three heteroatoms independently selected from oxygen, nitrogen, and sulfur. Examples of preferred five- and six-member heteroaryl groups include benzo[b]thienyl, chromenyl, furyl, imidazolyl, indazolyl, indolizinyl, indolyl, isobenzofuranyl, isoindolyl, isoquinolyl, isothiazolyl, isoxazolyl, naphthyridinyl, oxadiazolyt, oxazinyl, oxazolyl, phthalazinyl, pteridinyl, purinyl, pyranyl, pyrazinyl, pyrazolyl, pyridazinyl, pyridyl, pyrimidyl, pyrrolyl, quinolizinyl, quinolyl, quinoxalinyl, thiazolyl, thienyl, triazinyl, triazolyl, and xanthenyl.

The term “heterocycloalkyl” denotes a cycloalkyl system, wherein “cycloalkyl” is defined above, in which one or more of the ring carbon atoms are replaced with a heteroatom selected from the group consisting of nitrogen, oxygen, and sulfur. Examples of such heterocycloalkyl groups include azabicycloheptanyl, azetidinyl, benzazepinyl, 1,3-dihydroisoindolyl, indolinyl, tetrahydrofuryl, tetrahydroquinolinyl, tetrahydroisoquinolinyl, morpholinyl, piperazinyl, piperidyl, pyrrolidinyl, and, tetrahydro-2H-1,4-thiazinyl.

A cyclic group may be bonded to another group in more than one way. If no particular bonding arrangement is specified, then all possible arrangements are intended. For example, the term “pyridyl” includes 2-, 3-, or 4-pyridyl, and the term “thienyl” includes 2- or 3-thienyl.

The term “C₀-C₄” includes the embodiment where there are no carbons in a chain. Thus, for example, the groups “C₃-C₇ cycloalkyl-C₀-C₄ alkyl,” “C₆-C₁₄ aryl-C₀-C₄ alkyl,” “5-10-membered heteroaryl-C₀-C₄ alkyl,” and “C₆-C₁₄ aryl-C₀-C₄ alkylene-O—C₀-C₄ alkyl” include C₃-C₇ cycloalkyl, C₆-C₁₄ aryl, 5-10-membered heteroaryl, and C₆-C₁₄ aryl-O—C₀-C₄ alkyl, respectively.

The term “C₁-C₄ dialkylamino” refers to a dialkylamino group in which each alkyl group is independently a C₁-C₄ alkyl group.

This invention is also directed to:

a pharmaceutical composition for treating, for example, a disorder or condition that may be treated by antagonizing histamine-3 receptors, the composition comprising a compound of formula I as described above, and optionally a pharmaceutically acceptable carrier;

a method of treatment of a disorder or condition that may be treated by antagonizing histamine-3 receptors, the method comprising administering to a mammal in need of such treatment a compound of formula I as described above; and

a pharmaceutical composition for treating, for example, a disorder or condition selected from the group consisting of depression, mood disorders, schizophrenia, anxiety disorders, Alzheimer's disease, attention-deficit disorder (ADD), attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the composition comprising a compound of formula I as described above, and optionally a pharmaceutically acceptable carrier.

This invention is also directed to a method of treatment of a disorder or condition selected from the group consisting of the disorders or conditions listed in the preceding paragraph, the method comprising administering to a mammal in need of such treatment a compound of formula I as described above.

The histamine-3 (H3) receptor antagonists of the invention are useful for treating, in particular, ADD, ADHD, obesity, anxiety disorders and respiratory diseases. Respiratory diseases that may be treated by the present invention include adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

The pharmaceutical composition and method of this invention may also be used for preventing a relapse in a disorder or condition described in the previous paragraphs. Preventing such relapse is accomplished by administering to a mammal in need of such prevention a compound of formula I as described above.

The disclosed compounds may also be used as part of a combination therapy, including their administration as separate entities or combined in a single delivery system, which employs an effective dose of a histamine H3 antagonist compound of general formula I and an effective dose of a histamine H1 antagonist, such as cetirizine (Zyrtec™), for the treatment of allergic rhinitis, nasal congestion and allergic congestion.

The disclosed compounds may also be used as part of a combination therapy, including their administration as a separate entities or combined in a single delivery system, which employs an effective dose of a histamine H3 antagonist compound of general formula I and an effective dose of a neurotransmitter reuptake blocker. Examples of neurotransmitter reuptake blockers will include the serotonin-selective reuptake inhibitors (SSRI's) like sertraline (Zoloft™), fluoxetine (Prozac™), and paroxetine (Paxil™), or non-selective serotonin, dopamine or norepinephrine reuptake inhibitors for treating depression and mood disorders.

The compounds of the present invention may have optical centers and therefore may occur in different enantiomeric configurations. Formula I, as depicted above, includes all enantiomers, diastereomers, and other stereoisomers of the compounds depicted in structural formula I, as well as racemic and other mixtures thereof. Individual isomers can be obtained by known methods, such as optical resolution, optically selective reaction, or chromatographic separation in the preparation of the final product or its intermediate.

The present invention also includes isotopically labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I of this invention and prodrugs thereof can generally be prepared by carrying out the procedures disclosed in the Schemes and/or in the Examples and Preparations below, by substituting a readily available isotopically labeled reagent for a non-isotopically labeled reagent.

“Antagonizing histamine-3 (H3) receptors,” as used herein, refers to acting as a histamine-3 receptor antagonist.

A “unit dosage form” as used herein is any form that contains a unit dose of the compound of formula I. A unit dosage form may be, for example, in the form of a tablet or a capsule. The unit dosage form may also be in liquid form, such as a solution or suspension.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., depression) is 0.1 to 200 mg of the active ingredient per unit dose which could be administered, for example, 1 to 4 times per day.

Aerosol formulations for treatment of the conditions referred to above (e.g., attention deficit hyperactivity disorder) in the average human are preferably arranged so that each metered dose or “puff” of aerosol contains 20 μg to 1000 μg of the compound of the invention. The overall daily dose with an aerosol will be within the range 100 μg to 10 mg. Administration may be several times daily, for example 2, 3, 4 or 8 times, giving for example, 1, 2 or 3 doses each time.

In connection with the use of an active compound of this invention with a histamine H1 antagonist, preferably cetirizine, for the treatment of subjects possessing any of the above conditions, it is to be noted that these compounds may be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 95% by weight of the total composition, i.e., in amounts which are sufficient to provide the desired unit dosage and a histamine H1 antagonist, preferably cetirizine, is present in such dosage forms at concentration levels ranging from about 0.5% to about 95% by weight of the total composition, i.e., in amounts which are sufficient to provide the desired unit dosage.

A proposed daily dose of an active compound of this invention in the combination formulation (a formulation containing an active compound of this invention and a histamine H1 antagonist) for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

A proposed daily dose of a histamine H1 antagonist, preferably cetirizine, in the combination formulation for oral, parenteral or buccal administration to the average adult human for the treatment of the conditions referred to above is from about 0.1 mg to about 2000 mg, preferably from about 1 mg to about 200 mg of the histamine H1 antagonist per unit dose which could be administered, for example, 1 to 4 times per day.

A preferred dose ratio of cetirizine to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a histamine H1 antagonist, preferably cetirizine, preferably from about 1 mg to about 200 mg of cetirizine. Administration may be several times daily, for example 2, 3, 4 or 8 times, giving for example, 1, 2 or 3 doses each time.

As previously indicated, a histamine H1 antagonist, preferably cetirizine, in combination with compounds of formula I are readily adapted to therapeutic use as antidepressant agents. In general, these antidepressant compositions containing a histamine H1 antagonist, preferably cetirizine, and a compound of formula I are normally administered in dosages ranging from about 0.01 mg to about 100 mg per kg of body weight per day of a histamine H1 antagonist, preferably cetirizine, preferably from about 0.1 mg. to about 10 mg per kg of body weight per day of cetirizine; with from about 0.001 mg. to about 100 mg per kg of body weight per day of a compound of formula I, preferably from about 0.01 mg to about 10 mg per kg of body weight per day of a compound of formula I, although variations will necessarily occur depending upon the conditions of the subject being treated and the particular route of administration chosen.

In connection with the use of an active compound of this invention with a neurotransmitter re-uptake blocker, preferably sertraline, for the treatment of subjects possessing any of the above conditions, it is to be noted that these compounds may be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 95% by weight of the total composition, i.e., in amounts which are sufficient to provide the desired unit dosage and a neurotransmitter re-uptake blocker, preferably sertraline, is present in such dosage forms at concentration levels ranging from about 0.5% to about 95% by weight of the total composition, i.e., in amounts which are sufficient to provide the desired unit dosage.

A proposed daily dose of an active compound of this invention in the combination formulation (a formulation containing an active compound of this invention and a SSRI re-uptake inhibitor) for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

A proposed daily dose of a neurotransmitter re-uptake blocker, preferably sertraline, in the combination formulation for oral, parenteral or buccal administration to the average adult human for the treatment of the conditions referred to above is from about 0.1 mg to about 2000 mg, preferably from about 1 mg to about 200 mg of the neurotransmitter re-uptake blocker per unit dose which could be administered, for example, 1 to 4 times per day.

A preferred dose ratio of sertraline to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a neurotransmitter re-uptake blocker, preferably sertraline, preferably from about 1 mg to about 200 mg of sertraline. Administration may be several times daily, for example 2, 3, 4 or 8 times, giving for example, 1, 2 or 3 doses each time.

As previously indicated, a neurotransmitter re-uptake blocker, preferably sertraline, in combination with compounds of formula I are readily adapted to therapeutic use as antidepressant agents. In general, these antidepressant compositions containing a neurotransmitter re-uptake blocker, preferably sertraline, and a compound of formula I are normally administered in dosages ranging from about 0.01 mg to about 100 mg per kg of body weight per day of a neurotransmitter re-uptake blocker, preferably sertraline, preferably from about 0.1 mg. to about 10 mg per kg of body weight per day of sertraline; with from about 0.001 mg. to about 100 mg per kg of body weight per day of a compound of formula I, preferably from about 0.01 mg to about 10 mg per kg of body weight per day of a compound of formula I, although variations will necessarily occur depending upon the conditions of the subject being treated and the particular route of administration chosen.

Anxiety disorders include, for example, generalized anxiety disorder, panic disorder, PTSD, and social anxiety disorder. Mood adjustment disorders include, for example, depressed mood, mixed anxiety and depressed mood, disturbance of conduct, and mixed disturbance of conduct and depressed mood. Attention adjustment disorders include, for example, in addition to ADHD, attention-deficit disorders or other cognitive disorders due to general medical conditions. Psychotic disorders include, for example, schizoaffective disorders and schizophrenia; sleep disorders include, for example, narcolepsy and enuresis.

Examples of the disorders or conditions which may be treated by the compound, composition and method of this invention are also as follows: depression, including, for example, depression in cancer patients, depression in Parkinson's patients, post-myocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; dysthymia; phobias, including, for example, agoraphobia, social phobia or simple phobias; eating disorders, including, for example, anorexia nervosa or bulimia nervosa; chemical dependencies, including, for example, addictions to alcohol, cocaine, amphetamine and other psychostimulants, morphine, heroin and other opioid agonists, phenobarbital and other barbiturates, nicotine, diazepam, benzodiazepines and other psychoactive substances; Parkinson's diseases, including, for example, dementia in Parkinson's disease, neuroleptic-induced parkinsonism or tardive dyskinesias; headache, including, for example, headache associated with vascular disorders; withdrawal syndrome; age-associated learning and mental disorders; apathy; bipolar disorder; chronic fatigue syndrome; chronic or acute stress; conduct disorder; cyclothymic disorder; somatoform disorders such as somatization disorder, conversion disorder, pain disorder, hypochondriasis, body dysmorphic disorder, undifferentiated disorder, and somatoform NOS; incontinence; inhalation disorders; intoxication disorders; mania; oppositional defiant disorder; peripheral neuropathy; post-traumatic stress disorder; late luteal phase dysphoric disorder; specific developmental disorders; SSRI “poop out” syndrome, or a patient's failure to maintain a satisfactory response to SSRI therapy after an initial period of satisfactory response; and tic disorders including Tourette's disease.

As an example, the mammal in need of the treatment or prevention may be a human. As another example, the mammal in need of the treatment or prevention may be a mammal other than a human.

A compound of formula I that is basic in nature is capable of forming a wide variety of different salts with various inorganic and organic acids. The acid addition salts are readily prepared by treating the base compounds with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid salts of the active compound used in formulating the pharmaceutical composition of this invention that are basic in nature are those which form non-toxic acid addition salts, i.e., salts containing pharmacologically acceptable anions. Non-limiting examples of the salts include the acetate, benzoate, beta-hydroxybutyrate, bisulfate, bisulfite, bromide, butyne-1,4-dioate, caproate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogen phosphate, naphthalene-1-sulfonate, naphthalene-2-sulfonate, oxalate, phenylbutyrate, phenylpropionate, phosphate, phthalate, phenylacetate, propanesulfonate, propiolate, propionate, pyrophosphate, pyrosulfate, sebacate, suberate, succinate, sulfate, sulfite, sulfonate, tartrate, xylenesulfonate, acid phosphate, acid citrate, bitartrate, succinate, gluconate, saccharate, nitrate, methanesulfonate and pamoate [i.e., 1,1′-methylene-bis-(2-hydroxy-3-naphthoate)] salts.

(A) R¹ is methyl, R² is methyl; or

(B) R¹ and R² together with the nitrogen to which they are attached form the 5-membered pyrrolidine ring; or

(C) R¹ and R³ together with the nitrogen to which they are attached form a 5-membered pyrrolidine ring; or

(D) R¹ and R² together with the nitrogen to which they are attached form the 6-membered piperidine ring; or

(E) R¹ and R³ together with the nitrogen to which they are attached form the 6-membered piperidine ring: or

(F) R¹ and R² together with the nitrogen to which they are attached form the 5-membered pyrrolidine ring, and R^{2″} and R^{3″} together with the nitrogen to which they are attached form the 6-membered substituted piperizine or morpholine ring.

The most preferred embodiment of the present invention include the compounds of formula I in which R¹ and R² together with nitrogen to which they are attached form the 5-membered pyrrolidine ring.

Preferred embodiments of the present invention also include any combination of the foregoing embodiments (A)-(F).

Preferred compounds of formula I in accordance with the present invention are the following:

2,5-Bis-pyrrolidin-1-ylmethyl-1H-indole.

5-Morpholin-4-ylmethyl-2-pyrrolidin-1-ylmethyl-1H-indole.

2-Pyrrolidin-1-ylmethyl-5-thiomorpholin-4-ylmethyl-1H-indole.

1-[4-(2-Pyrrolidin-1-ylmethyl-1H-indol-5-ylmethyl)-piperazin-1-yl]-ethanone.

6-[4-(2-Pyrrolidin-1-ylmethyl-1H-indol-5-ylmethyl)-piperazin-1-yl]-nicotinonitrile.

2-[Ethyl-(2-pyrrolidin-1-ylmethyl-1H-indol-5-ylmethyl)-amino]-ethanol.

1-(2-Pyrrolidin-1-ylmethyl-1H-indol-5-ylmethyl)-piperidin-4-ol.

1-[4-Phenyl-1-(2-pyrrolidin-1-ylmethyl-1H-indol-5-ylmethyl)-piperidin-4-yl]-ethanone.

Isopropyl-(2-pyrrolidin-1-ylmethyl-1H-indol-5-ylmethyl)-(1,3,5-trimethyl-1H-pyrazol-4-ylmethyl)-amine.

N-Butyl-N-methyl-N′-(2-pyrrolidin-1-ylmethyl-1H-indol-5-ylmethyl)-ethane-1,2-diamine.

5-(4-Pyrimidin-2-yl-[1,4]diazepan-1-ylmethyl)-2-pyrrolidin-1-ylmethyl-1H-indole.

Cyclopropyl-(2-pyrrolidin-1-ylmethyl-1H-indol-5-ylmethyl)-(1,3,5-trimethyl-1H-pyrazol-4-ylmethyl)-amine.

5-[4-(1-Methyl-1H-imidazol-2-ylmethyl)-piperazin-1-ylmethyl]-2-pyrrolidin-1-ylmethyl-1H-indole.

[4-(2-Pyrrolidin-1-ylmethyl-1H-indol-5-ylmethyl)-piperazin-1-yl]-acetic acid methyl ester.

Methyl-(3-methyl-pyridin-2-ylmethyl)-(2-pyrrolidin-1-ylmethyl-1H-indol-5-ylmethyl)-amine.

(2-Furan-2-yl-ethyl)-(2-pyrrolidin-1-ylmethyl-1H-indol-5-ylmethyl)-amine.

5-[4-(2-Methyl-thiazol-4-ylmethyl)-piperazin-1-ylmethyl]-2-pyrrolidin-1-ylmethyl-1H-indole.

1-Phenyl-4-(2-pyrrolidin-1-ylmethyl-1H-indol-5-ylmethyl)-piperazin-2-one.

Methyl-(5-propyl-1H-pyrazol-3-ylmethyl)-(2-pyrrolidin-1-ylmethyl-1H-indol-5-ylmethyl)-amine.

2-Cyclopropyl-6-(2-pyrrolidin-1-ylmethyl-1H-indol-5-ylmethyl)-5,6,7,8-tetrahydro-pyrido[4,3-d]pyrimidine.

(4-Phenyl-thiazol-2-ylmethyl)-(2-pyrrolidin-1-ylmethyl-1H-indol-5-ylmethyl)-amine.

1-(3,5-Dimethyl-phenyl)-4-[(2-pyrrolidin-1-ylmethyl-1H-indol-5-yl)methyl]-piperazin-2-one.

(6-Methyl-imidazo[1,2-a]pyridin-2-ylmethyl)-(2-pyrrolidin-1-ylmethyl-1H-indol-5-ylmethyl)-amine.

(3-Pyrazol-1-yl-benzyl)-(2-pyrrolidin-1-ylmethyl-1H-indol-5-ylmethyl)-amine.

(1-Methyl-1H-imidazol-2-ylmethyl)-(2-pyrrolidin-1-ylmethyl-1H-indol-5-ylmethyl)-amine.

(2-Imidazo[1,2-a]pyridin-2-yl-ethyl)-(2-pyrrolidin-1-ylmethyl-1H-indol-5-ylmethyl)-amine.

(7-Methyl-imidazo[1,2-a]pyridin-2-ylmethyl)-(2-pyrrolidin-1-ylmethyl-1H-indol-5-ylmethyl)-amine.

1-(4-Fluoro-benzyl)-4-(2-pyrrolidin-1-ylmethyl-1H-indol-5-ylmethyl)piperazin-2-one.

Methyl-(2-pyrrolidin-1-ylmethyl-1H-indol-5-ylmethyl)-quinoxalin-2-ylmethyl-amine.

5-(4-Morpholin-4-yl-piperidin-1-ylmethyl)-2-pyrrolidin-1-ylmethyl-1H-indole.

(5-Methyl-imidazo[1,2-a]pyridin-2-ylmethyl)-(2-pyrrolidin-1-ylmethyl-1H-indol-5-ylmethyl)-amine.

Imidazo[1,2-a]pyridin-2-ylmethyl-(2-pyrrolidin-1-ylmethyl-1H-indol-5-ylmethyl)-amine.

(2-Methoxy-2-methyl-propyl)-(2-pyrrolidin-1-ylmethyl-1H-indol-5-ylmethyl)-amine.

(1,5-Dimethyl-1H-pyrazol-4-ylmethyl)-(2-pyrrolidin-1-ylmethyl-1H-indol-5-ylmethyl)-amine.

[2-(3,5-Dimethyl-pyrazol-1-yl)-ethyl]-methyl-(2-pyrrolidin-1-ylmethyl-1H-indol-5-ylmethyl)-amine.

6-(2-Pyrrolidin-1-ylmethyl-1H-indol-5-ylmethyl)-2-trifluoromethyl-5,6,7,8-tetrahydro-pyrido[4,3-d]pyrimidine.

Methyl-(4-methyl-1H-imidazol-2-ylmethyl)-(2-pyrrolidin-1-ylmethyl-1H-indol-5-ylmethyl)-amine.

Methyl-[2-(4-methyl-thiazol-5-yl)-ethyl]-(2-pyrrolidin-1-ylmethyl-1H-indol-5-ylmethyl)-amine.

[2-(3,5-Dimethyl-pyrazol-1-yl)-ethyl]-(2-pyrrolidin-1-ylmethyl-1H-indol-5-ylmethyl)-amine.

2-Pyridin-2-yl-6-(2-pyrrolidin-1-ylmethyl-1H-indol-5-ylmethyl)-5,6,7,8-tetrahydro-pyrido[4,3-d]pyrimidine.

Methyl-(2-pyrrolidin-1-ylmethyl-1H-indol-5-ylmethyl)-(tetrahydro-pyran-4-ylmethyl)-amine.

2-Methoxy-6-(2-pyrrolidin-1-ylmethyl-1H-indol-5-ylmethyl)-5,6,7,8-tetrahydro-pyrido[4,3-d]pyrimidine.

Methyl-pyrimidin-4-ylmethyl-(2-pyrrolidin-1-ylmethyl-1H-indol-5-ylmethyl)-amine

2-Pyridin-4-yl-6-(2-pyrrolidin-1-ylmethyl-1H-indol-5-ylmethyl)-5,6,7,8-tetrahydro-pyrido[4,3-d]pyrimidine.

(5-Phenyl-isoxazol-3-ylmethyl)-(2-pyrrolidin-1-ylmethyl-1H-indol-5-ylmethyl)-amine.

Benzothiazol-2-ylmethyl-(2-pyrrolidin-1-ylmethyl-1H-indol-5-ylmethyl)-amine.

(4-Methyl-thiazol-2-ylmethyl)-(2-pyrrolidin-1-ylmethyl-1H-indol-5-ylmethyl)-amine.

(3-Phenyl-[1,2,4]oxadiazol-5-ylmethyl)-(2-pyrrolidin-1-ylmethyl-1H-indol-5-ylmethyl)-amine.

Methyl-(3-phenyl-[1,2,4]oxadiazol-5-ylmethyl)-(2-pyrrolidin-1-ylmethyl-1H-indol-5-ylmethyl)-amine.

4-(2-Pyrrolidin-1-ylmethyl-1H-indol-5-ylmethyl)-2,3,4,5-tetrahydro-benzo[f][1,4]oxazepine.

2-Pyrrolidin-1-ylmethyl-5-(4-thiophen-3-ylmethyl-piperazin-1-ylmethyl)-1H-indole.

Methyl-(5-phenyl-[1,3,4]oxadiazol-2-ylmethyl)-(2-pyrrolidin-1-ylmethyl-1H-indol-5-ylmethyl)-amine.

Methyl-(4-phenyl-thiazol-2-ylmethyl)-(2-pyrrolidin-1-ylmethyl-1H-indol-5-ylmethyl)-amine.

Methyl-(5-methyl-1H-pyrazol-3-ylmethyl)-(2-pyrrolidin-1-ylmethyl-1H-indol-5-ylmethyl)-amine.

(2-Pyrrolidin-1-ylmethyl-1H-indol-5-ylmethyl)-(4,5,6,7-tetrahydro-benzothiazol-2-ylmethyl)-amine.

5-((R)-2-Morpholin-4-ylmethyl-pyrrolidin-1-ylmethyl)-2-pyrrolidin-1-ylmethyl-1H-indole.

5-((S)-3-Morpholin-4-yl-pyrrolidin-1-ylmethyl)-2-pyrrolidin-1-ylmethyl-1H-indole.

5-[(S)-2-(4-Methyl-piperazin-1-ylmethyl)-pyrrolidin-1-ylmethyl]-2-pyrrolidin-1-ylmethyl-1H-indole.

(2-Phenyl-oxazol-4-ylmethyl)-(2-pyrrolidin-1-ylmethyl-1H-indol-5-ylmethyl)-amine.

5-[(S)-3-(2-Ethoxy-ethoxy)-pyrrolidin-1-ylmethyl]-2-pyrrolidin-1-ylmethyl-1H-indole.

2-[4-(2-Pyrrolidin-1-ylmethyl-1H-indol-5-ylmethyl)-piperazin-1-yl]-nicotinamide.

(R)-4-Methanesulfonyl-1-(2-pyrrolidin-1-ylmethyl-1H-indol-5-ylmethyl)-piperazine-2-carboxylic acid methyl ester.

5-(3-Morpholin-4-yl-azetidin-1-ylmethyl)-2-pyrrolidin-1-ylmethyl-1H-indole.

5-((S)-3-Propoxy-pyrrolidin-1-ylmethyl)-2-pyrrolidin-1-ylmethyl-1H-indole.

5-[4-(6-Methyl-pyridin-2-yl)-[1,4]diazepan-1-ylmethyl]-2-pyrrolidin-1-ylmethyl-1H-indole.

5-[(R)-3-(2-Ethoxy-ethoxy)-pyrrolidin-1-ylmethyl]-2-pyrrolidin-1-ylmethyl-1H-indole.

N,N-Dimethyl-2-[1-(2-pyrrolidin-1-ylmethyl-1H-indol-5-ylmethyl)-piperidin-4-yloxy]-acetamide.

2-Methyl-1-{4-[(2-pyrrolidin-1-ylmethyl-1H-indol-5-ylmethyl)-amino]-piperidin-1-yl}-propan-1-one.

5-[(R)-3-(3-Methoxy-propoxy)-pyrrolidin-1-ylmethyl]-2-pyrrolidin-1-ylmethyl-1H-indole.

[2-(4-Chloro-phenoxy)-5-fluoro-benzyl]-methyl-(2-pyrrolidin-1-ylmethyl-1H-indol-5-ylmethyl)-amine.

N,N-Dimethyl-2-[(R)-1-(2-pyrrolidin-1-ylmethyl-1H-indol-5-ylmethyl)-pyrrolidin-3-yloxy]-acetamide.

N-tert-Butyl-2-[4-(2-pyrrolidin-1-ylmethyl-1H-indol-5-ylmethyl)-piperazin-1-yl]-nicotinamide.

5-[(R)-2-(3-Ethyl-[1,2,4]oxadiazol-5-yl)-pyrrolidin-1-ylmethyl]-2-pyrrolidin-1-ylmethyl-1H-indole.

5-[(R)-2-(3-Methyl-[1,2,4]oxadiazol-5-yl)-pyrrolidin-1-ylmethyl]-2-pyrrolidin-1-ylmethyl-1H-indole.

5-[(S)-3-(2-Methoxy-ethoxy)-pyrrolidin-1-ylmethyl]-2-pyrrolidin-1-ylmethyl-1H-indole.

Ethyl-pyridin-3-ylmethyl-(2-pyrrolidin-1-ylmethyl-1H-indol-5-ylmethyl)-amine.

5-[(S)-2-(3-Ethyl-[1,2,4]oxadiazol-5-yl)-pyrrolidin-1-ylmethyl]-2-pyrrolidin-1-ylmethyl-1H-indole.

5-[(S)-2-(3-Methyl-[1,2,4]oxadiazol-5-yl)-pyrrolidin-1-ylmethyl]-2-pyrrolidin-1-ylmethyl-1H-indole.

5-[(S)-3-(3-Methoxy-propoxy)-pyrrolidin-1-ylmethyl]-2-pyrrolidin-1-ylmethyl-1H-indole.

1-{4-[(2-Pyrrolidin-1-ylmethyl-1H-indol-5-ylmethyl)-amino]-piperidin-1-yl}propan-1-one.

5-[4-(3-Methyl-pyridin-2-yl)-[1,4]diazepan-1-ylmethyl]-2-pyrrolidin-1-ylmethyl-1H-indole.

### DETAILED DESCRIPTION OF THE INVENTION

The compound of formula (I) according to the invention may be prepared by the general procedure shown in Scheme 1.

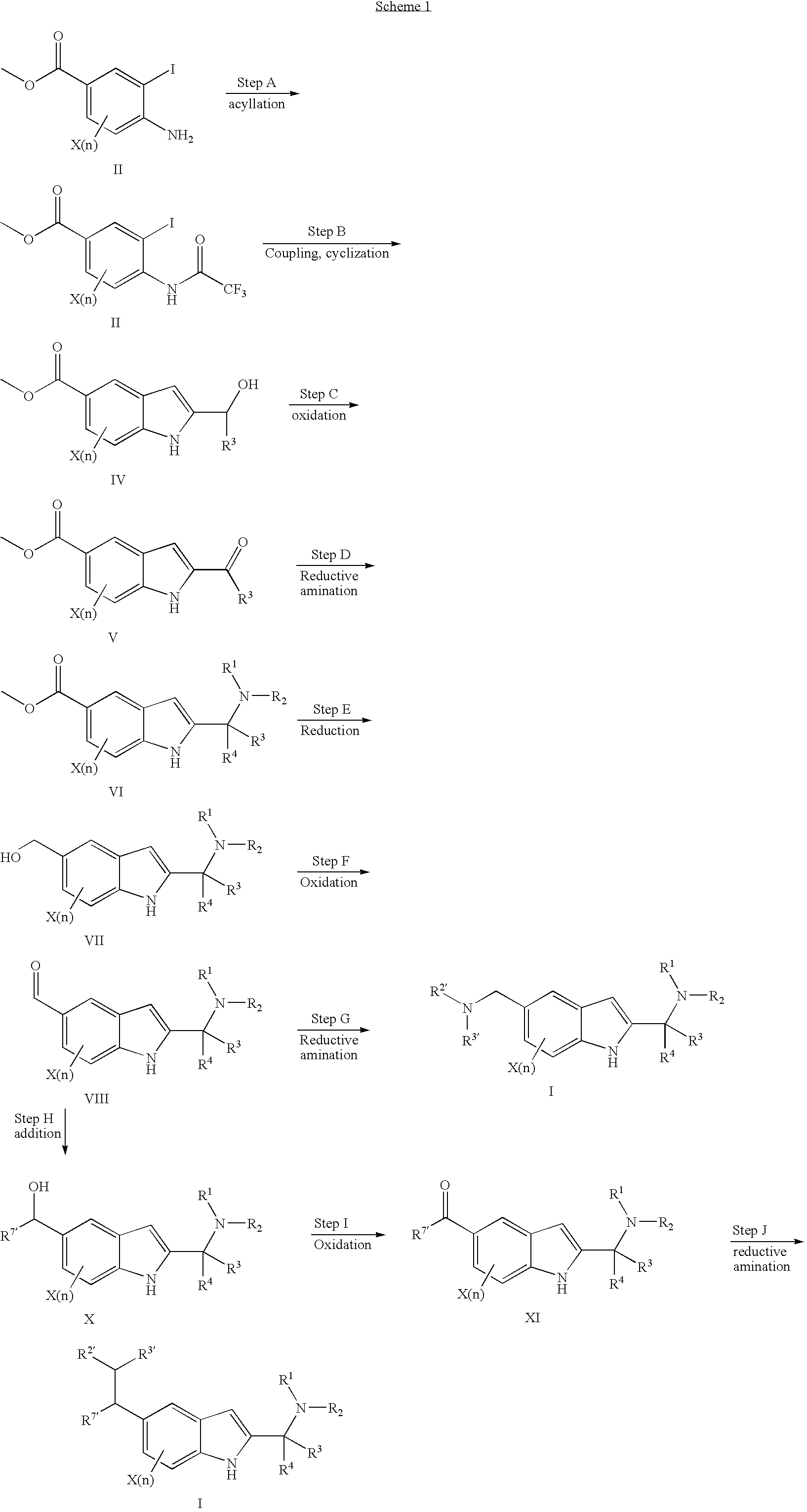

In Scheme 1, compounds of the formula (I) are prepared as follows.

### Step A

Intermediate of the formula II may be reacted with an anhydride or and acid chloride, where trifluoroacetic anhydride is prefered, in a reaction inert solvent, where preferred solvents are chlorinated solvents such as dichloromethane or 1,2-dichloroethane at the reflux temperature of the solvent employed to give a compound of the formula III.

### Step B

Intermediate of the formula III may be converted to intermediates of the formula IV using well-established chemistry that is in the literature. One such approach uses copper(I) iodide, Pd(Ph₃P)₂Cl₂ and triethyl amine (e.g., see Fagnola, M. C.; Candiani, I; Visentin, G; Cabri, W.;Zarini, F.; Mongelli, N.: and Bedeschi, A. in Tetrahedron Letters, 1997, 38, 1997). In such a conversion, the intermediate of the formula III and the appropriate alkyne are combined in a reaction inert solvent. Suitable solvents include, among others, tetrahydrofuran (THF) and dioxane, dimethyl formamide (DMF), and dimethyl acetamide, where the preferred solvent is DMF. To this mixture is added CuI, Pd(PPh₃)₂Cl₂ and a base, such as, but not limited to tetramethylguanidine or triethylamine. The reaction can generally be carried out at atmospheric pressure and at temperatures ranging from about rt to the reflux temperature of the solvent employ, typically at rt −60° C. and most preferably at 60° C. to give a compound of the formula IV.

### Step C

Intermediate of the formula IV may be oxidized using standard oxidation conditions that appear in the literature to give intermediates of the formula V. This can be accomplished, for example, using excess activated manganese(IV) oxide, in reaction inert solvent, where chloroform, methylene chloride, or methanol are suitable, at a reaction temperature from about room temperature to the reflux temperature of the solvent employed, where room temperature is preferred to give an intermediate of the formula V.

### Step D

Intermediates of general formula V may then be reacted with primary or secondary amines of general formula HNR¹R² (XIII), where R¹ and R² are as defined in the specification. This can be accomplished, for example, using a procedure referred to as reductive amination which is a method well known to those skilled in the art. This method may be conducted in a single, concerted process (e.g., see A. F. Abdel-Magid, C. A. Maryanoff and K. G. Carson in Tetrahedron Letters, 1990, 39:5595-5598). In such conversions, the carbonyl compound of formula V and the appropriate amine of formula X are combined in a reaction inert solvent and treated with reagents like sodium cyanoborohydride or sodium triacetoxyborohydride. Suitable solvents include, among others, tetrahydrofuran (THF) and 1,2-dichloroethane (DCE) and the reactions may be conducted with or without the addition of an organic acid (e.g., acetic acid).

Alternatively, the conversion of compounds of formula V to compounds of formula VI can be completed using two or more individual steps, involving the initial formation of an imine intermediate such as XIV, followed by reduction of the C═N double bond to generate VI.

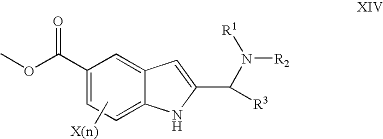

For example, the intermediate of formula V and the amine XIII of formula HNR¹R² can be combined in the presence of a dehydrating reagent in a reaction neutral solvent like benzene, toluene, methanol or ethanol and stirred for a prescribed amount of time until the reaction is judged to be completed. Such dehydrating reagents include, for example, p-toluenesulfonic acid, titanium(IV)chloride, titanium(IV) isopropoxide or molecular sieves. The reaction can be conducted within the range of about 0° C. to about the boiling point of the solvent employed and at pressures of about one to about three atmospheres. The intermediate imine XIV so obtained can then be reduced with a variety of reagents and under a variety of conditions familiar to one skilled in the art, including the use of hydrogen gas in the presence of a catalyst like palladium on carbon (Pd/C) or platinum on carbon (Pt/C), as well as with sodium borohydride, sodium (triacetoxy)borohydride, sodium cyanoborohydride and the like. The use of hydrogen as the reducing agent is often conducted in a reaction inert solvent such as methanol, ethanol, THF, 1,4-dioxane and similar solvents at a pressure of about one atmosphere to a pressure of about 5 atmospheres of hydrogen and typically at a temperature from about room temperature to a temperature that is below the boiling point of the solvent employed. When using the hydride reagents, the choice of solvent can be made from, but not limited to, methanol, ethanol, isopropanol, 1,4-dioxane, THF and the like. The reaction can generally be carried out at atmospheric pressure and at temperatures ranging from about −40° C. to about the boiling temperature of the solvent employed, typically at 0-40° C. and most preferably at room temperature.

### Step E

The ester of an intermediate of the general formula VI is reduced with an appropriate reduction reagent such as, but not limited to, lithium aluminum hydride, or sodium borohydride and aluminum trichloride in diglyme, where lithium aluminum hydride is preferred. The reaction is normally effected in an aprotic solvent, such as tetrahydrofuran, or diethyl ether at a reaction temperature from about 0 C to the reflux temperature of the solvent employed, yielding the an intermediated of the general formula VII.

### Step F

Alcohols of the formula VII may be oxidized using standard oxidation conditions that appear in the literature to give intermediates of the formula VIII. This can be accomplished, for example, using excess activated manganese(IV) oxide, in reaction inert solvent, where chloroform, methylene chloride, or methanol are suitable, at a reaction temperature from about room temperature to the reflux temperature of the solvent employed, where room temperature is preferred to give an intermediate of the formula VIII.

### Step G

Intermediates of general formula VIII may then be reacted with primary or secondary amines of general formula HNR^{2″}R^{3″} (XV), where R^{2″} and R^{3″} are as defined in the specification. This can be accomplished, for example, using a procedure referred to as reductive amination which is a method well known to those skilled in the art. This method may be conducted in a single, concerted process (e.g., see A. F. Abdel-Magid, C. A. Maryanoff and K. G. Carson in Tetrahedron Letters, 1990, 39:5595-5598). In such conversions, the carbonyl compound of formula VIII and the appropriate amine of formula XV are combined in a reaction inert solvent and treated with reagents like sodium cyanoborohydride or sodium triacetoxyborohydride. Suitable solvents include, among others, tetrahydrofuran (THF) and 1,2-dichloroethane (DCE) and the reactions may be conducted with or without the addition of an organic acid (e.g., acetic acid) to give a compound of the formula I

Alternatively, the conversion of compounds of formula VIII to compounds of formula I can be completed using two or more individual steps, involving the initial formation of an imine intermediate such as XVI, followed by reduction of the C═N double bond to generate a compound of the formula I.

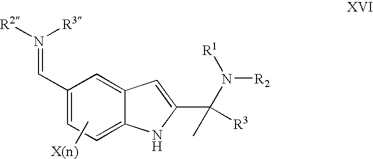

For example, the intermediate of formula VIII and the amine XV of formula HNR^{2″}R^{3″} (XV) can be combined in the presence of a dehydrating reagent in a reaction neutral solvent like benzene, toluene, methanol or ethanol and stirred for a prescribed amount of time until the reaction is judged to be completed. Such dehydrating reagents include, for example, p-toluenesulfonic acid, titanium(IV)chloride, titanium(IV) isopropoxide or molecular sieves. The reaction can be conducted within the range of about 0° C. to about the boiling point of the solvent employed and at pressures of about one to about three atmospheres. The intermediate imine XVI so obtained can then be reduced with a variety of reagents and under a variety of conditions familiar to one skilled in the art, including the use of hydrogen gas in the presence of a catalyst like palladium on carbon (Pd/C) or platinum on carbon (Pt/C), as well as with sodium borohydride, sodium (triacetoxy)borohydride, sodium cyanoborohydride and the like. The use of hydrogen as the reducing agent is often conducted in a reaction inert solvent such as methanol, ethanol, THF, 1,4-dioxane and similar solvents at a pressure of about one atmosphere to a pressure of about 5 atmospheres of hydrogen and typically at a temperature from about room temperature to a temperature that is below the boiling point of the solvent employed. When using the hydride reagents, the choice of solvent can be made from, but not limited to, methanol, ethanol, isopropanol, 1,4-dioxane, THF and the like. The reaction can generally be carried out at atmospheric pressure and at temperatures ranging from about −40° C. to about the boiling temperature of the solvent employed, typically at 0-40° C. and most preferably at room temperature to give a compound of the formula I.

### Step H

Aldehydes of the general formula VIII may be converted to intermediates of the formula VIV by reaction with an organo metal reagent (Grignard reagents) of the general formula R⁷″MetalX, where R^{7″} is defined in the specification. Grignard reagents maybe, but not limited to magnesium and lithium (e.g., R^{7″}MgBr, R^{7″}Li). The reaction is done in a reaction inert solvent, such a THF. The reaction can generally be carried out at atmospheric pressure and at temperatures ranging from about −78° C. to about the boiling temperature of the solvent employed, typically at −78 to rt ° C. and most preferably at about rt to give a compound of the formula VIV.

### Step I

Alcohols of the formula VIV may be oxidized using standard oxidation conditions that appear in the literature to give intermediates of the formula X. This can be accomplished, for example, using excess activated manganese(IV) oxide, in reaction inert solvent, where chloroform, methylene chloride, or methanol are suitable, at a reaction temperature from about room temperature to the reflux temperature of the solvent employed, where room temperature is preferred to give an intermediate of the formula X.

### Step J

Intermediates of general formula X may then be reacted with primary or secondary amines of general formula HNR^{2″}R^{3″} (XV), where R^{2″} and R^{3″} are as defined in the specification. This can be accomplished, for example, using a procedure referred to as reductive amination which is a method well known to those skilled in the art. This method may be conducted in a single, concerted process (e.g., see A. F. Abdel-Magid, C. A. Maryanoff and K. G. Carson in Tetrahedron Letters, 1990, 39:5595-5598). In such conversions, the carbonyl compound of formula X and the appropriate amine of formula XV are combined in a reaction inert solvent and treated with reagents like sodium cyanoborohydride or sodium triacetoxyborohydride. Suitable solvents include, among others, tetrahydrofuran (THF) and 1,2-dichloroethane (DCE) and the reactions may be conducted with or without the addition of an organic acid (e.g., acetic acid) to give a compound of the formula I

Alternatively, the conversion of compounds of formula X to compounds of formula I can be completed using two or more individual steps, involving the initial formation of an imine intermediate such as XVII, followed by reduction of the C═N double bond to generate a compound of the formula I.

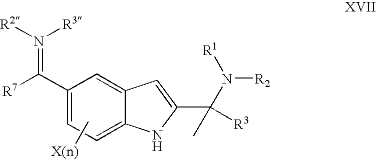

For example, the intermediate of formula X and the amine XV of formula HNR^{2″}R^{3″} (XV) can be combined in the presence of a dehydrating reagent in a reaction neutral solvent like benzene, toluene, methanol or ethanol and stirred for a prescribed amount of time until the reaction is judged to be completed. Such dehydrating reagents include, for example, p-toluenesulfonic acid, titanium(IV)chloride, titanium(IV) isopropoxide or molecular sieves. The reaction can be conducted within the range of about 0° C. to about the boiling point of the solvent employed and at pressures of about one to about three atmospheres. The intermediate imine XVII so obtained can then be reduced with a variety of reagents and under a variety of conditions familiar to one skilled in the art, including the use of hydrogen gas in the presence of a catalyst like palladium on carbon (Pd/C) or platinum on carbon (Pt/C), as well as with sodium borohydride, sodium (triacetoxy)borohydride, sodium cyanoborohydride and the like. The use of hydrogen as the reducing agent is often conducted in a reaction inert solvent such as methanol, ethanol, THF, 1,4-dioxane and similar solvents at a pressure of about one atmosphere to a pressure of about 5 atmospheres of hydrogen and typically at a temperature from about room temperature to a temperature that is below the boiling point of the solvent employed. When using the hydride reagents, the choice of solvent can be made from, but not limited to, methanol, ethanol, isopropanol, 1,4-dioxane, THF and the like. The reaction can generally be carried out at atmospheric pressure and at temperatures ranging from about −40° C. to about the boiling temperature of the solvent employed, typically at 0-40° C. and most preferably at room temperature to give a compound of the formula I.

In the intermediates and examples below the following terms are intended to have the following, general meaning:

bs: broad singlet

d.e.: diatomaceous earth, filter agent

DMF: dimethyformamide

LRMS: low resolution mass spectrometry

calcd; calculated

d; doublet (spectral)

EtOAc: ethyl acetate

J: coupling constant (in NMR)

LAH: lithium aluminum hydride

m: multiplet (in NMR)

Min: minute(s)

m/z: mass to charge ratio (in mass spectrometry)

obsd: observed

Rf: retention factor (in chromatography)

Rt: retention time (in chromatography)

rt: room temperature

s: singlet (NMR), second(s)

t: triplet

TFA: trifluoroacetic acid

TFAA: trifluoroacetic anhydride

THF: tetrahydrofuran

tic: thin layer chromatography

Solvents were purchased and used without purification. Yields were calculated for material judged homogenous by thin layer chromatography and NMR. Thin layer chromatography was performed on Merck Kieselgel 60 F 254 plates eluting with the solvents indicated, visualized by a 254 nm UV lamp, and stained with either an aqueous KMnO₄ solution or an ethanolic solution of 12-molybdophosphoric acid. Flash column chromatography was performed with using either pre-packed Biotage® or ISCO® columns using the size indicated. Nuclear magnetic resonance (NMR) spectra were acquired on a Unity 400 or 500 at 400 MHz or 500 MHz for ¹H, respectively, and 100 MHz or 125 MHz for ¹³C NMR, respectively. Chemical shifts for proton ¹H NMR spectra are reported in parts per million relative to the singlet of CDCl₃ at 7.24 ppm. Chemical shifts for ¹³C NMR spectra are reported in parts per million downfield relative to the centerline of the triplet of CDCl₃ at 77.0 ppm. Mass spectra analyses were performed on a APCI Gilson 215, micromass ZMD (50% Acetonitrile /50% water) spectrometer.

Reactions under microwave conditions were done using 2-5 mL round bottom vials, fitted with septa. The vials containing the reactants were inserted into the reaction chamber of a EMRYS™ Creator microwave apparatus (maximum power of 300 W) from Personal Chemistry Inc., 25 Birch St., Bldg C, Suite 304, Milford, Mass. 01757 and heated to the appropriate temperature for a the prescribed period of time. HPLC was performed according to the following methods:

General Procedure A: To the respective amines (0.1 mmol, 2 equiv) weighed into a 2-dram vial dissolved in 0.4 mL of DCE as added the the aldehyde intermediate 6 (11.4 mg, equiv) as a solution dissolved in 0.4 ml of DCE and acetic acid (0.006 ml, 0.1 mmol, 2 equiv). The reaction was shaken at room temperature for 5 hr, and then Na(OAc)₃BH (˜32 mg, 0.15 mmol, 3 equiv) was added neat in one portion. The resulting reaction mixture was shaken at room temperature over night. LRMS analysis of crude reaction mixture indicated product formation. The reactions were quenched by partitioning the samples between 2.5 ml of methylene chloride and 1.5 ml of aqueous NaOH (1 M), vortexed and the organics were extracted and load onto Silicycle SCX SPE cartridge (6-ml). Repeat extraction 2×. Change vials and elute with 5 ml of MeOH. Switch to tared vials and elute with 7.5 ml of 1 N TEA in MeOH. The solvents were removed under reduced pressure and the residual was purified by HPLC using method indicated.

General Procedure B: To the respective amines salts (0.1 mmol, 2 equiv) weighed into a 2-dram vial was dissolved in 0.1 mL of DCE. The aldehyde intermediate VIII (13.2 mg, 0.05 mmol, 1 equiv) was added as a solution dissolved in 0.5 ml of DCE and a solution of triethylamine (0.014 ml, 0.1 mmol, 2 equiv, in 0.1 mL of DCE). The reaction was shaken at room temperature overnight, and then Na(OAc)3BH (˜21 mg, 0.1 mmol, 2 equiv) was added neat in one portion. The resulting reaction mixture was shaken at room temperature for ˜3 hours. LRMS analysis of crude reaction mixture indicated product formation. The reactions were quenched by partitioning the samples between 2.5 ml of methylene chloride and 1.5 ml of aqueous NaOH (1 M), vortexed and the organics were extracted and load onto Silicycle SCX SPE cartridge (6-ml). Repeat extraction 2×. Change vials and elute with 5 ml of MeOH. Switch to tared vials and elute with 7.5 ml of 1 N TEA in MeOH. The solvents were removed under reduced pressure and the residual was purified by HPLC using method indicated.

Purification Method A: Preparative conditions (Waters 600 & Waters 2767 Sample Manager); Column: Waters Xterra PrepMS C₁₈, 5 μm, 30×100 mm steel column, part # 186001120, solvent A—0.1% Trifluoroacetic acid/water; solvent B—Acetonitile; volume of injection: 1100 μL; time 0.0, 100% solvent A, 0% solvent B, flow 20; time 2.0, 100% solvent A, 0% solvent B, flow 20; time 12.0, 0% solvent A, 100% solvent B, flow 20; time 14.0, 0% solvent A, 100% solvent B, flow 20; time 14.1, 100% solvent A, 0% solvent B, flow 20; time 19, 100% solvent A, 0% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550.

Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

The following intermediates may be prepared by the procedures described above:

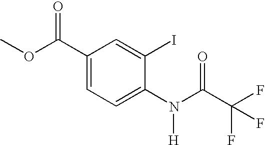

### 3-Iodo-4-(2,2,2-trifluoro-acetylamino)-benzoic acid methyl ester

To a stirring solution of 3-iodobenzoate (6.0 g, 21.7 mmol) in 80 mL of methylene chloride at 0 C (ice water bath) was added pyridine (1.9 mL, 23.8 mmol) follow by trifluoroacetic anhydride (3.3 mL, 23.8 mmol). Reaction was then allowed to warm to rt and stirred over night. After 12 hr the reaction was quenched with aqueous 1M HCl and then diluted with methylene chloride. The organic layer was washed with a saturated solution of NaHCO₃, dried over MgSO₄, filtered through a fritted funnel and then concentrated under reduced pressure to give a white solid. Purification of this material was accomplished by flash column chromatography using an ISCO column (330 g) eluting with 25% EtOAc/hexanes. The product containing fractions were collected and concentrated to give the title compound (7.2 g, 89% yield) as a white solid.

Rf=0.54 (20% EtOAc/hexanes); 400 MHz 1H NMR (CDCl3) δ 8.51 (s, 1H), 8.50 (s, 1H), 8.36 (d, J=8.7 Hz, 1H), 8.08 (dd, J=8.3. 1.3 Hz, 1H), 3.93 (s, 3H); LRMS m/z Calcd for C10H7F3INO3 372.9; obsd LRMS APCI (M−1) m/z 372.

### Intermediate 2

### 2-Hydroxymethyl-1H-indole-5-carboxylic acid methyl ester

To a stirring solution 3-Iodo-4-(2,2,2-trifluoro-acetylamino)-benzoic acid methyl ester, intermediate 1 (0.2 g, 0.54 mmol) in DMF (5 mL) was added propargyl alchol (47 uL, 0.8 mmol), Pd(Ph₃P)₂Cl₂ (19 mg, 0.027 mmol), Cul (10 mg, 0.054 mmol), and triethyl amine (0.37 mL, 2.7 mmol). The reaction was then heated to 60 C (oil bath) for 5 hr and then the reaction was cooled to rt. The reaction was then concentrated under reduced pressure and purified. Purification of this material was accomplished by flash column chromatography using a 40 g ISCO column, eluting was a gradient of 30%, 60% EtOAc/hexanes. The product containing fractions were collected and concentrated to yield the title compound (100 mg, 95% yield) as a brown oil.

Rf=0.38 (70% EtOAc/hexanes); Anal Cald for C₁₁H₉NO₃:: Exact Mass: 205.07; obsd LRMS APCI (M−1) m/z 204.

### Intermediate 3

### 2-Formyl-1H-indole-5-carboxylic acid methyl ester

To a stirring solution of intermediate 2, 2-hydroxymethyl-1H-indole-5-carboxylic acid methyl ester (4.7 g, 23.1 mmol) in 70 mL of chloroform was added in two portions MnO₂ (12.5 g, 115.4 mmol). After 14 hr the reaction was filtered through a plug of celite and then concentrated under reduced pressure to give the title compound (4.0 g, 85% yield) as a brown oil. This material was used without further purification.

Rf=0.21 (20% EtOAc/hexanes); Anal Clcd for C₁₁H₁₁NO₃: Exact Mass: 203.06; obsd LRMS APCI (M−1) m/z 202.

### Intermediate 4

### 2-Pyrrolidin-1-ylmethyl-1H-indole-5-carboxylic acid methyl ester

To a stirring solution of intermediate 3, 2-formyl-1H-indole-5-carboxylic acid methyl ester (0.75 g, 3.7 mmol) in methylene chloride (20 mL) was added pyrrolidine (1.5 mL, 18.5 mmol), followed by acetic acid (50 uL) and sodium triacetoxyborohydride (0.94 g, 4.4 mmol). After 5 hr the reaction was quenched with a saturated aqueous solution of NaHCO₃ and then the layers were separated. The aqueous layer was extracted with 3:1 CHCl₃:isopropyl alcohol and then the combined organic layers were dried over Na₂SO₄, filtered through a fritted funnel and concentrated under reduced pressure. Purification of this material was accomplished by flash column chromatography using 120 g ISCO column, eluting with a gradient of 2%, 5%, 10%, 20% MeOH/CH₂Cl₂. The product containing fractions were collected and concentrated under reduced pressure to give the title compound with acetic acid (0.55 g, 57% yield) as a brown oil.

Rf=0.31 (10% MeOH/CH₂Cl₂); Anal Clcd for C₁₅H₁₈N₂O₂ Exact Mass: 258.14; obsd LRMS APCI m/z 259.1 (m+1).

### Intermediate 5

### (2-Pyrrolidin-1-ylmethyl-1H-indol-5-yl)-methanol

To a stirring solution of intermediate 4, 2-pyrrolidin-1-ylmethyl-1H-indole-5-carboxylic acid methyl ester (5.0 g, 19.7 mmol) in THF (100 mL) at 0 C (ice water bath) was added a THF solution of lithium aluminum hydride (69 mL, 68.9 mmol, 1M THF). After 1 hr 15 the reaction was quenched cold using the Feiser method (H₂O, 15% NaOH, H₂O). The resulting slurry was filtered through a plug of Celite. The plug was washed with 3:1 CH₃Cl:isopropyl alcohol. The combined organic layers were concentrated under reduced pressure. Purification of this material was accomplished by flash column chromatography, using a 120 g ISCO column, elution with a gradient of 5%, 10%, 20%, 30% MeOH/CH₂Cl₂ with 0.2% NH₄OH. The product containing fractions were collected and concentrated to give the title compound (3.5 g, 78% yield) as a semi solid, brown in color.

Rf=0.18 (10% MeOH/CH₂Cl₂ w/0.1% NH₄OH); Anal Clcd for C₁₄H₁₈N₂O: Exact Mass: 230.14; obsd LRMS APCI m/z 231 (m+1).

### Intermediate 6

### 2-Pyrrolidin-1-ylmethyl-1H-indole-5-carbaldehyde

To a stirring solution of intermediate 5, (2-pyrrolidin-1-ylmethyl-1H-indol-5-yl)-methanol (12.5 g, 54.3 mmol) in methylene chloride (100 mL) was added in portions MnO₂ (58.9 g, 542 mmol). After 16 hours the reaction was filtered through a plug of celite, and then the filter pad was washed with methylene chloride, and methanol. The filtrates were concentrated under reduced pressure to give the title compound as a brown gummy oil. This material was used without further purification.

Rf=0.32 (10% MeOH/CH₂Cl₂); Anal Clcd for C₁₄H₁₆N₂O: Exact Mass: 228.13; obsd LRMS APCI m/z 229 (m+1).

### EXAMPLE 1

### 2,5-Bis-pyrrolidin-1-ylmethyl-1H-indole

To a stirring solution of intermediate 6, 2-pyrrolidin-1-ylmethyl-1H-indole-5-carbaldehyde (100 mg, 0.43 mmol) in methylene chloride at rt. To this solution was added acetic acid (50 uL), pyrrolidine (183 uL, 2.2 mmol) followed by sodium triacetoxy borohydride (111 mg, 0.53 mmol). After 3 hr the reaction was complete by TLC analysis. Reaction was quenched with NaHCO₃, and extracted with isopropyl alcohol:chloroform (3:1). The organic layer was dried over MgSO₄, filtered and concentrated under reduced pressure. Purification of this material was accomplished by flash column chromatography using a 12 g ISCO column, eluting with a gradient of 5%, 10%, 15%, MeOH/CH₂Cl₂ with 0.1% NH₄H. The product-containing fractions were collected and concentrated to give the title compound (66 mg, 53% yield). Rt=0.15 (20% MeOH/CH₂Cl₂ with 0.1% NH₄OH); LRMS m/z Calcd for C18 H25 N3 283.4; obsd LRMS APCI (M+1) m/z. The HCl salt was made by dissolving the free base in EtOAc and adding 1 eq of HCl (2M diethyl ether). The solid was filtrated and dryed under reduced pressure yield a tan solid.

### EXAMPLE 2

### 2-Pyrrolidin-1-ylmethyl-5-thiomorpholin-4-ylmethyl-1H-indole

Reaction was conducted using the reaction conditions described is Example 1. Using thiomorpholine as the amine.

Rf=0.25 (20% MeOH/CH₂Cl₂ with 0.1% NH₄OH); LRMS m/z Calcd for C18 H25 N3 S 315.5; obsd LRMS APCI (M+1) m/z 316.

### EXAMPLE 3

### 1-[4-(2-Pyrrolidin-1-ylmethyl-1H-indol-5-ylmethyl)-piperazin-1-yl]-ethanone

Reaction was conducted using the reaction conditions described is Example 1. Using 1-acetylpiperizine as the amine.

Rf=0.24 (20% MeOH/CH₂Cl₂ with 0.1% NH₄OH); LRMS m/z Calcd for C20 H28 N4 O 340.5; obsd LRMS APCI (M+1) m/z 341.

### EXAMPLE 4

### 5-Morpholin-4-ylmethyl-2-pyrrolidin-1-ylmethyl-1H-indole

Reaction was conducted using the general procedure A; purification was accomplished using purification method A); isolated weight=14 mg; HPLC purity (%) at 220 nM=92; Rt=1.27; LRMS m/z Calcd for C18 H25 N3 O 299.4; obsd LRMS APCI (M+1) m/z 300.2.

### EXAMPLE 6

### 2-[Ethyl-(2-pyrrolidin-1-ylmethyl-1H-indol-5-ylmethyl)-amino]-ethanol

Reaction was conducted using the general procedure A; purification was accomplished using purification method A); isolated weight=11.6 mg; HPLC purity (%) at 220 nM=94; Rt=1.31; LRMS m/z Calcd for C18 H27 N3 O 301.4; obsd LRMS APCI (M+1) m/z 302.2.

### EXAMPLE 7

### -1-(2-Pyrrolidin-1-ylmethyl-1H-indol-5-ylmethyl)-piperidin-4-ol

Reaction was conducted using the general procedure A; purification was accomplished using purification method A; isolated weight=10.9 mg; HPLC purity (%) at 220 nM=92; Rt=1.29; LRMS m/z Calcd for C19 H27 N3 O 313.4; obsd LRMS APCI (M+1) m/z 314.2.

### EXAMPLE 8

### 1-[4-Phenyl-1-(2-pyrrolidin-1-ylmethyl-1H-indol-5-ylmethyl)-piperidin-4-yl]-ethanone

Reaction was conducted using the general procedure A; purification was accomplished using purification method A; isolated weight=12.9 mg; HPLC purity (%) at 220 nM=100; Rt=2.06; LRMS m/z Calcd for C27 H33 N3 O 415.6; obsd LRMS APCI (M+1) m/z 416.2.

### EXAMPLE 9

### General Procedure A

### Isopropyl-(2-pyrrolidin-1-ylmethyl-1H-indol-5-ylmethyl)-(1,3,5-trimethyl-1H-pyrazol-4-ylmethyl)-amine

Reaction was conducted using the general procedure A; purification was accomplished using purification method A; isolated weight=6.21 mg; HPLC purity (%) at 220 nM=90; Rt=1.83; LRMS m/z Calcd for C24 H35 N5 393.6; obsd LRMS APCI (M+1) m/z 394.3.

### EXAMPLE 10

### N-Butyl-N-methyl-N′-(2-pyrrolidin-1-ylmethyl-1H-indol-5-ylmethyl)-ethane-1,2-diamine

Reaction was conducted using the general procedure A; purification was accomplished using purification method A; isolated weight=1.8 mg; HPLC purity (%) at 220 nM=94; Rt=1.6; LRMS m/z Calcd for C21 H34 N4 342.5; obsd LRMS APCI (M+1) m/z 343.3.

### EXAMPLE 11

### -5-(4-Pyrimidin-2-yl-[1,4]diazepan-1-ylmethyl)-2-pyrrolidin-1-ylmethyl-1H-indole

Reaction was conducted using the general procedure A; purification was accomplished using purification method A; isolated weight=7.5 mg; HPLC purity (%) at 220 nM=100; Rt=1.75; LRMS m/z Calcd for C23 H30 N6 390.5; obsd LRMS APCI (M+1) m/z 391.2.

### EXAMPLE 12

### Cyclopropyl-(2-pyrrolidin-1-ylmethyl-1H-indol-5-ylmethyl)-(1,3,5-trimethyl-1H-pyrazol-4-ylmethyl)-amine

Reaction was conducted using the general procedure A; purification was accomplished using purification method A; isolated weight=8.9 mg; HPLC purity (%) at 220 nM=95; Rt=1.79; LRMS m/z Calcd for C24 H33 N5 391.6; obsd LRMS APCI (M+1) m/z 392.2.

### EXAMPLE 13

### 5-[4-(1-Methyl-1H-imidazol-2-ylmethyl)-piperazin-1-ylmethyl]-2-pyrrolidin-1-ylmethyl-1H-indole

Reaction was conducted using the general procedure A; purification was accomplished using purification method A; isolated weight=7 mg; HPLC purity (%) at 220 nM=100; Rt=1.16; LRMS m/z Calcd for C23 H32 N6 392.5; obsd LRMS APCI (M+1) m/z 393.3.

### EXAMPLE 14

### [4-(2-Pyrrolidin-1-ylmethyl-1H-indol-5-ylmethyl)-piperazin-1-yl]-acetic acid methyl ester

Reaction was conducted using the general procedure A; purification was accomplished using purification method A; isolated weight=0.7 mg; HPLC purity (%) at 220 nM=100; Rt=1.48; LRMS m/z Calcd for C21 H30 N4 O2 370.5; obsd LRMS APCI (M+1) m/z 371.2.

### EXAMPLE 15

### General Procedure A

### Methyl-(3-methyl-pyridin-2-ylmethyl)-(2-pyrrolidin-1-ylmethyl-1H-indol-5-ylmethyl)-amine

Reaction was conducted using the general procedure A; purification was accomplished using purification method A; isolated weight=4.9 mg; HPLC purity (%) at 220 nM=91; Rt=1.88; LRMS m/z Calcd for C22 H28 N4 348.5; obsd LRMS APCI (M+1) m/z 349.2.

### EXAMPLE 16

### (2-Furan-2-yl-ethyl)-(2-pyrrolidin-1-ylmethyl-1H-indol-5-ylmethyl)-amine

Reaction was conducted using the general procedure A; purification was accomplished using purification method A; isolated weight=4.9 mg; HPLC purity (%) at 220 nM=100; Rt=1.85; LRMS m/z Calcd for C20 H25 N3 O 323.4; obsd LRMS APCI (M+1) m/z 324.2.

### EXAMPLE 17

### 5-[4-(2-Methyl-thiazol-4-ylmethyl)-piperazin-1-ylmethyl]-2-pyrrolidin-1-ylmethyl-1H-indole

Reaction was conducted using the general procedure A; purification was accomplished using purification method A; isolated weight=8.5 mg; HPLC purity (%) at 220 nM=100; Rt=1.62; LRMS m/z Calcd for C23 H31 N5 S 409.6; obsd LRMS APCI (M+1) m/z 410.2.

### EXAMPLE 18

### 1-Phenyl-4-(2-pyrrolidin-1-ylmethyl-1H-indol-5-ylmethyl)-piperazin-2-one

Reaction was conducted using the general procedure A; purification was accomplished using purification method A; isolated weight=7.12 mg; HPLC purity (%) at 220 nM=100; Rt=2; LRMS m/z Calcd for C22 H31 N5 365.5; obsd LRMS APCI (M+1) m/z 366.3.

### EXAMPLE 19

### Methyl-(5-propyl-1H-pyrazol-3-ylmethyl)-(2-pyrrolidin-1-ylmethyl-1H-indol-5-ylmethyl)-amine

Reaction was conducted using the general procedure A; purification was accomplished using purification method A; isolated weight=7 mg; HPLC purity (%) at 220 nM=100; Rt=1.77; LRMS m/z Calcd for C24 H28 N4 O 388.5; obsd LRMS APCI (M+1) m/z 389.2.

### EXAMPLE 20

### 2-Cyclopropyl-6-(2-pyrrolidin-1-ylmethyl-1H-indol-5-ylmethyl)-5,6,7,8-tetrahydro-pyrido[4,3-d]pyrimidine

Reaction was conducted using the general procedure A; purification was accomplished using purification method A; isolated weight=7.3 mg; HPLC purity (%) at 220 nM=92; Rt=1.79; LRMS m/z Calcd for C24 H29 N5 387.5; obsd LRMS APCI (M+1) m/z 388.2.

### EXAMPLE 21

### General Procedure A

### (4-Phenyl-thiazol-2-ylmethyl)-(2-pyrrolidin-1-ylmethyl-1H-indol-5-ylmethyl)-amine

Reaction was conducted using the general procedure A; purification was accomplished using purification method A; isolated weight=1.65 mg; HPLC purity (%) at 220 nM=90; Rt=1.85; LRMS m/z Calcd for C24 H26 N4 S 402.6; obsd LRMS APCI (M+1) m/z 403.2.

### EXAMPLE 22

### 1-(3,5-Dimethyl-phenyl)-4-[(2-pyrrolidin-1-ylmethyl-1H-indol-5-yl)methyl]-piperazin-2-one

Reaction was conducted using the general procedure A; purification was accomplished using purification method A; isolated weight=3.2 mg; HPLC purity (%) at 220 nM=90; Rt=1.71; LRMS m/z Calcd for C26 H32 N4 O 416.6; obsd LRMS APCI (M+1) m/z 417.3.

### EXAMPLE 23

### (6-Methyl-imidazo[1,2-a]pyridin-2-ylmethyl)-(2-pyrrolidin-1-ylmethyl-1H-indol-5-ylmethyl)-amine

Reaction was conducted using the general procedure A; purification was accomplished using purification method A; isolated weight=5.9 mg; HPLC purity (%) at 220 nM=100; Rt=1.62; LRMS m/z Calcd for C23 H27 N5 373.5; obsd LRMS APCI (M+1) m/z 374.0.

### EXAMPLE 24

### (3-Pyrazol-1-yl-benzyl)-(2-pyrrolidin-1-ylmethyl-1H-indol-5-ylmethyl)-amine

Reaction was conducted using the general procedure A; purification was accomplished using purification method A; isolated weight=2.8 mg; HPLC purity (%) at 220 nM=100; Rt=1.65; LRMS m/z Calcd for C24 H27 N5 385.5; obsd LRMS APCI (M+1) m/z 386.3.

### EXAMPLE 25

### (1-Methyl-1H-imidazol-2-ylmethyl)-(2-pyrrolidin-1-ylmethyl-1H-indol-5-ylmethyl)-amine

Reaction was conducted using the general procedure A; purification was accomplished using purification method A; isolated weight=3.5 mg; HPLC purity (%) at 220 nM=100; Rt=1.37; LRMS m/z Calcd for C19 H25 N5 323.4; obsd LRMS APCI (M+1) m/z 324.2.

### EXAMPLE 26

### (2-Imidazo[1,2-a]pyridin-2-yl-ethyl)-(2-pyrrolidin-1-ylmethyl-1H-indol-5-ylmethyl)-amine

Reaction was conducted using the general procedure A; purification was accomplished using purification method A; isolated weight=3.4 mg; HPLC purity (%) at 220 nM=92; Rt=1.58; LRMS m/z Calcd for C23 H27 N5 373.5; obsd LRMS APCI (M+1) m/z 374.0.

### EXAMPLE 27

### (7-Methyl-imidazo[1,2-a]pyridin-2-ylmethyl)-(2-pyrrolidin-1-ylmethyl-1H-indol-5-ylmethyl)-amine

Reaction was conducted using the general procedure A; purification was accomplished using purification method A; isolated weight=5 mg; HPLC purity (%) at 220 nM=100; Rt=1.62; LRMS m/z Calcd for C23 H27 N5 373.5; obsd LRMS APCI (M+1) m/z 374.0.

### EXAMPLE 28

### 1-(4-Fluoro-benzyl)-4-(2-pyrrolidin-1-ylmethyl-1H-indol-5-ylmethyl)-piperazin-2-one

Reaction was conducted using the general procedure A; purification was accomplished using purification method A; isolated weight=10 mg; HPLC purity (%) at 220 nM=90; Rt=2.04; LRMS m/z Calcd for C25 H29 F N4 O 420.5; obsd LRMS APCI (M+1) m/z 421.0.

### EXAMPLE 29

### Methyl-(2-pyrrolidin-1-ylmethyl-1H-indol-5-ylmethyl)-quinoxalin-2-ylmethyl-amine

Reaction was conducted using the general procedure A; purification was accomplished using purification method A; isolated weight=2.31 mg; HPLC purity (%) at 220 nM=100; Rt=1.5; LRMS m/z Calcd for C24 H27 N5 385.5; obsd LRMS APCI (M+1) m/z 386.3.

### EXAMPLE 30

### 5-(4-Morpholin-4-yl-piperidin-1-ylmethyl)-2-pyrrolidin-1-ylmethyl-1H-indole

Reaction was conducted using the general procedure A; purification was accomplished using purification method A; isolated weight=4.1 mg; HPLC purity (%) at 220 nM=90; Rt=1.4; LRMS m/z Calcd for C23 H34 N4 O 382.5; obsd LRMS APCI (M+1) m/z 383.0.

### EXAMPLE 31

### (5-Methyl-imidazo[1,2-a]pyridin-2-ylmethyl)-(2-pyrrolidin-1-ylmethyl-1H-indol-5-ylmethyl)-amine

Reaction was conducted using the general procedure A; purification was accomplished using purification method A; isolated weight=7.3 mg; HPLC purity (%) at 220 nM=100; Rt=1.6; LRMS m/z Calcd for C23 H27 N5 373.5; obsd LRMS APCI (M+1) m/z 374.0.

### EXAMPLE 32

### Imidazo[1,2-a]pyridin-2-ylmethyl-(2-pyrrolidin-1-ylmethyl-1H-indol-5-ylmethyl)-amine

Reaction was conducted using the general procedure A; purification was accomplished using purification method A; isolated weight=3.4 mg; HPLC purity (%) at 220 nM=100; Rt=1.69; LRMS m/z Calcd for C20 H27 N5 337.5; obsd LRMS APCI (M+1) m/z 338.0.

### EXAMPLE 33

### (2-Methoxy-2-methyl-propyl)-(2-pyrrolidin-1-ylmethyl-1H-indol-5-ylmethyl)-amine

Reaction was conducted using the general procedure A; purification was accomplished using purification method A; isolated weight=3.9 mg; HPLC purity (%) at 220 nM=100; Rt=1.52; LRMS m/z Calcd for C22 H25 N5 359.5; obsd LRMS APCI (M+1) m/z 360.0.

### EXAMPLE 34

### (1,5-Dimethyl-1H-pyrazol-4-ylmethyl)-(2-pyrrolidin-1-ylmethyl-1H-indol-5-ylmethyl)-amine

Reaction was conducted using the general procedure A; purification was accomplished using purification method A; isolated weight=8.1 mg; HPLC purity (%) at 220 nM=100; Rt=1.65; LRMS m/z Calcd for C19 H29 N3 O 315.5; obsd LRMS APCI (M+1) m/z 316.0.

### EXAMPLE 35

### [2-(3,5-Dimethyl-pyrazol-1-yl)-ethyl]-methyl-(2-pyrrolidin-1-ylmethyl-1H-indol-5-ylmethyl)-amine

Reaction was conducted using the general procedure A; purification was accomplished using purification method A; isolated weight=3.2 mg; HPLC purity (%) at 220 nM=100; Rt=1.5; LRMS m/z Calcd for C22 H31 N5 365.5; obsd LRMS APCI (M+1) m/z 366.0.

### EXAMPLE 36

### 6-(2-Pyrrolidin-1-ylmethyl-1H-indol-5-ylmethyl)-2-trifluoromethyl-5,6,7,8-tetrahydro-pyrido[4,3-d]pyrimidine

Reaction was conducted using the general procedure A; purification was accomplished using purification method A; isolated weight=9.8 mg; HPLC purity (%) at 220 nM=100; Rt=1.85; LRMS m/z Calcd for C22 H24 F3 N5 415.5; obsd LRMS APCI (M+1) m/z 416.0.

### EXAMPLE 37

### Methyl-(4-methyl-1H-imidazol-2-ylmethyl)-(2-pyrrolidin-1-ylmethyl-1H-indol-5-ylmethyl)-amine

Reaction was conducted using the general procedure A; purification was accomplished using purification method A; isolated weight=4.9 mg; HPLC purity (%) at 220 nM=100; Rt=1.46; LRMS m/z Calcd for C20 H27 N5 337.5; obsd LRMS APCI (M+1) m/z 338.0.

### EXAMPLE 38

### Methyl-[2-(4-methyl-thiazol-5-yl)-ethyl]-(2-pyrrolidin-1-ylmethyl-1H-indol-5-ylmethyl)-amine

Reaction was conducted using the general procedure A; purification was accomplished using purification method A; isolated weight=7.4 mg; HPLC purity (%) at 220 nM=100; Rt=1.65; LRMS m/z Calcd for C21 H28 N4 S 368.5; obsd LRMS APCI (M+1) m/z 369.0.

### EXAMPLE 39

### [2-(3,5-Dimethyl-pyrazol-1-yl)-ethyl]-(2-pyrrolidin-1-ylmethyl-1H-indol-5-ylmethyl)-amine

Reaction was conducted using the general procedure A; purification was accomplished using purification method A; isolated weight=8 mg; HPLC purity (%) at 220 nM=100; Rt=1.88; LRMS m/z Calcd for C21 H29 N5 351.5; obsd LRMS APCI (M+1) m/z 352.0.

### EXAMPLE 40

### 2-Pyridin-2-yl-6-(2-pyrrolidin-1-ylmethyl-1H-indol-5-ylmethyl)-5,6,7,8-tetrahydro-pyrido[4,3-d]pyrimidine

Reaction was conducted using the general procedure A; purification was accomplished using purification method A; isolated weight=2.1 mg; HPLC purity (%) at 220 nM=100; Rt=1.52; LRMS m/z Calcd for C26 H28 N6 424.5; obsd LRMS APCI (M+1) m/z 425.0.

### EXAMPLE 41

### General Procedure A

### Methyl-(2-pyrrolidin-1-ylmethyl-1H-indol-5-ylmethyl)-(tetrahydro-pyran-4-ylmethyl)-amine

Reaction was conducted using the general procedure A; purification was accomplished using purification method A; isolated weight=8.3 mg; HPLC purity (%) at 220 nM=100; Rt=1.63; LRMS m/z Calcd for C21 H31 N3 O 341.5; obsd LRMS APCI (M+1) m/z 342.0.

### EXAMPLE 42

### 2-Methoxy-6-(2-pyrrolidin-1-ylmethyl-1H-indol-5-ylmethyl)-5,6,7,8-tetrahydro-pyrido[4,3-d]pyrimidine

Reaction was conducted using the general procedure A; purification was accomplished using purification method A; isolated weight=10.3 mg; HPLC purity (%) at 220 nM=100; Rt=1.56; LRMS m/z Calcd for C22 H27 N5 O 377.5; obsd LRMS APCI (M+1) m/z 378.0.

### EXAMPLE 43

### Methyl-pyrimidin-4-ylmethyl-(2-pyrrolidin-1-ylmethyl-1H-indol-5-ylmethyl)-amine

Reaction was conducted using the general procedure A; purification was accomplished using purification method A; isolated weight=2.2 mg; HPLC purity (%) at 220 nM=100; Rt=1.56; LRMS m/z Calcd for C20 H25 N5 335.5; obsd LRMS APCI (M+1) m/z 336.0.

### EXAMPLE 44

### 2-Pyridin-4-yl-6-(2-pyrrolidin-1-ylmethyl-1H-indol-5-ylmethyl)-5,6,7,8-tetrahydro-pyrido[4,3-d]pyrimidine

Reaction was conducted using the general procedure A; purification was accomplished using purification method A; isolated weight=3.4 mg; HPLC purity (%) at 220 nM=100; Rt=1.56; LRMS m/z Calcd for C26 H28 N6 424.5; obsd LRMS APCI (M+1) m/z 425.0.

### EXAMPLE 45

### (5-Phenyl-isoxazol-3-ylmethyl)-(2-pyrrolidin-1-ylmethyl-1H-indol-5-ylmethyl)-amine

Reaction was conducted using the general procedure A; purification was accomplished using purification method A; isolated weight=9.3 mg; HPLC purity (%) at 220 nM=92; Rt=2.27; LRMS m/z Calcd for C24 H26 N4 O 386.5; obsd LRMS APCI (M+1) m/z 387.0.

### EXAMPLE 46

### Benzothiazol-2-ylmethyl-(2-pyrrolidin-1-ylmethyl-1H-indol-5-ylmethyl)-amine

Reaction was conducted using the general procedure A; purification was accomplished using purification method A; isolated weight=8.9 mg; HPLC purity (%) at 220 nM=95; Rt=2.08; LRMS m/z Calcd for C22 H24 N4 S 376.5; obsd LRMS APCI (M+1) m/z 377.0.

### EXAMPLE 47

### (4-Methyl-thiazol-2-ylmethyl)-(2-pyrrolidin-1-ylmethyl-1H-indol-5-ylmethyl)-amine

Reaction was conducted using the general procedure A; purification was accomplished using purification method A; isolated weight=1.7 mg; HPLC purity (%) at 220 nM=; Rt=1.4; LRMS m/z Calcd for C19 H24 N4 S 340.5; obsd LRMS APCI (M+1) m/z 341.0.

### EXAMPLE 48

### (3-Phenyl-[1,2,4]oxadiazol-5-ylmethyl)-(2-pyrrolidin-1-ylmethyl-1H-indol-5-ylmethyl)-amine

Reaction was conducted using the general procedure A; purification was accomplished using purification method A; isolated weight=11.4 mg; HPLC purity (%) at 220 nM=92; Rt=2.11; LRMS m/z Calcd for C23 H25 N5 O 387.5; obsd LRMS APCI (M+1) m/z 388.0.

### EXAMPLE 49

### Methyl-(3-phenyl-[1,2,4]oxadiazol-5-ylmethyl)-(2-pyrrolidin-1-ylmethyl-1H-indol-5-ylmethyl)-amine

Reaction was conducted using the general procedure A; purification was accomplished using purification method A; isolated weight=9.8 mg; HPLC purity (%) at 220 nM=100; Rt=1.71; LRMS m/z Calcd for C24 H27 N5 O 401.5; obsd LRMS APCI (M+1) m/z 402.0.

### EXAMPLE 50

### 4-(2-Pyrrolidin-1-ylmethyl-1H-indol-5-ylmethyl)-2,3,4,5-tetrahydro-benzo[f][1,4]oxazepin

Reaction was conducted using the general procedure A; purification was accomplished using purification method A; isolated weight=100 mg; HPLC purity (%) at 220 nM=; Rt=1.96; LRMS m/z Calcd for C23 H27 N3 O 361.5; obsd LRMS APCI (M+1) m/z 362.0.

### EXAMPLE 51

### 2-Pyrrolidin-1-ylmethyl-5-(4-thiophen-3-ylmethyl-piperazin-1-ylmethyl)-1H-indole

Reaction was conducted using the general procedure A; purification was accomplished using purification method A; isolated weight=5.9 mg; HPLC purity (%) at 220 nM=100; Rt=1.6; LRMS m/z Calcd for C23 H30 N4 S 394.6; obsd LRMS APCI (M+1) m/z 395.0.

### EXAMPLE 52

### Methyl-(5-phenyl-[1,3,4]oxadiazol-2-ylmethyl)-(2-pyrrolidin-1-ylmethyl-1H-indol-5-ylmethyl)amine

Reaction was conducted using the general procedure A; purification was accomplished using purification method A; isolated weight=12.4 mg; HPLC purity (%) at 220 nM=100; Rt=1.92; LRMS m/z Calcd for C24 H27 N5 O 401.5; obsd LRMS APCI (M+1) m/z 402.0.

### EXAMPLE 53

### Methyl-(4-phenyl-thiazol-2-ylmethyl)-(2-pyrrolidin-1-ylmethyl-1H-indol-5-ylmethyl)-amine

Reaction was conducted using the general procedure A; purification was accomplished using purification method A; isolated weight=15.3 mg; HPLC purity (%) at 220 nM=100; Rt=2.06; LRMS m/z Calcd for C25 H28 N4 S 416.6; obsd LRMS APCI (M+1) m/z 417.0.

### EXAMPLE 54

### General Procedure A

### Methyl-(5-methyl-1H-pyrazol-3-ylmethyl)-(2-pyrrolidin-1-ylmethyl-1H-indol-5-ylmethyl)-amine

Reaction was conducted using the general procedure A; purification was accomplished using purification method A; isolated weight=4.1 mg; HPLC purity (%) at 220 nM=100; Rt=2.12; LRMS m/z Calcd for C22 H28 N4 S 380.6; obsd LRMS APCI (M+1) m/z 381.0.

### EXAMPLE 55

### (2-pyrrolidin-1-ylmethyl-1H-indol-5-ylmethyl)-(4,5,6,7-tetrahydro-benzothiazol-2-ylmethyl)-amine

Reaction was conducted using the general procedure A; purification was accomplished using purification method A; isolated weight=6.8 mg; HPLC purity (%) at 220 nM=90; Rt=1.71; LRMS m/z Calcd for C20 H27 N5 337.5; obsd LRMS APCI (M+1) m/z 338.0.

### EXAMPLE 56

### 5-((R)-2-Morpholin-4-ylmethyl-pyrrolidin-1-ylmethyl)-2-pyrrolidin-1-ylmethyl-1H-indole

Reaction was conducted using the general procedure A; purification was accomplished using purification method A; isolated weight=10.2 mg; HPLC purity (%) at 220 nM=100; Rt=0.96; LRMS m/z Calcd for C23 H34 N4 O 382.5; obsd LRMS APCI (M+1) m/z 383.0.

### EXAMPLE 57

### 5-((S)-3-Morpholin-4-yl-pyrrolidin-1-ylmethyl)-2-pyrrolidin-1-ylmethyl-1H-indole

Reaction was conducted using the general procedure A; purification was accomplished using purification method A; isolated weight=4.4 mg; HPLC purity (%) at 220 nM=100; Rt=0.91; LRMS m/z Calcd for C22 H32 N4 O 368.5; obsd LRMS APCI (M+1) m/z 369.0.

### EXAMPLE 58

### 5-[(S)-2-(4-Methyl-piperazin-1-ylmethyl)-pyrrolidin-1-ylmethyl]-2-pyrrolidin-1-ylmethyl-1H-indole

Reaction was conducted using the general procedure A; purification was accomplished using purification method A; isolated weight=6 mg; HPLC purity (%) at 220 nM=100; Rt=0.95; LRMS m/z Calcd for C24 H37 N5 395.6; obsd LRMS APCI (M+1) m/z 396.0.

### EXAMPLE 59

### (2-Phenyl-oxazol-4-ylmethyl)-(2-pyrrolidin-1-ylmethyl-1H-indol-5-ylmethyl)-amine

Reaction was conducted using the general procedure A; purification was accomplished using purification method A; isolated weight=7.2 mg; HPLC purity (%) at 220 nM=90; Rt=1.44; LRMS m/z Calcd for C24 H26 N4 O 386.5; obsd LRMS APCI (M+1) m/z 387.0.

### EXAMPLE 60

### 5-[(S)-3-(2-Ethoxy-ethoxy)-pyrrolidin-1-ylmethyl]-2-pyrrolidin-1-ylmethyl-1H-indole

Reaction was conducted using the general procedure A; purification was accomplished using purification method A; isolated weight=8.6 mg; HPLC purity (%) at 220 nM=100; Rt=1.18; LRMS m/z Calcd for C22 H33 N3 O2 371.5; obsd LRMS APCI (M+1) m/z 372.0.

### EXAMPLE 61

### 2-[4-(2-Pyrrolidin-1-ylmethyl-1H-indol-5-ylmethyl)-piperazin-1-yl]-nicotinamide

Reaction was conducted using the general procedure A; purification was accomplished using purification method A; isolated weight=1.4 mg; HPLC purity (%) at 220 nM=100; Rt=1.06; LRMS m/z Calcd for C24 H30 N6 O 418.5; obsd LRMS APCI (M+1) m/z 419.0.

### EXAMPLE 62

### (R)-4-Methanesulfonyl-1-(2-pyrrolidin-1-ylmethyl-1H-indol-5-ylmethyl)-piperazine-2-carboxylic acid methyl ester.

Reaction was conducted using the general procedure A; purification was accomplished using purification method A; isolated weight=3.6 mg; HPLC purity (%) at 220 nM=100; Rt=1; LRMS m/z Calcd for C21 H30 N4 O4 S 434.6; obsd LRMS APCI (M+1) m/z 404 (lose of -OMe).

### EXAMPLE 63

### 5-(3-Morpholin-4-yl-azetidin-1-ylmethyl)-2-pyrrolidin-1-ylmethyl-1H-indole

Reaction was conducted using the general procedure A; purification was accomplished using purification method A; isolated weight=7.3 mg; HPLC purity (%) at 220 nM=100; Rt=0.98; LRMS m/z Calcd for C21 H30 N4 O 354.5; obsd LRMS APCI (M+l) m/z 355.0.

### EXAMPLE 64

### 5-((S)-3-Propoxy-pyrrolidin-1-ylmethyl)-2-pyrrolidin-1-ylmethyl-1H-indole

Reaction was conducted using the general procedure A; purification was accomplished using purification method A; isolated weight=7.2 mg; HPLC purity (%) at 220 nM=92; Rt=1.27; LRMS m/z Calcd for C21 H31 N3 O 341.5; obsd LRMS APCI (M+1) m/z 342.0.

### EXAMPLE 65

### 5-[4-(6-Methyl-pyridin-2-yl)-[1,4]diazepan-1-ylmethyl]-2-pyrrolidin-1-ylmethyl-1H-indole

Reaction was conducted using the general procedure A; purification was accomplished using purification method A; isolated weight=7.4 mg; HPLC purity (%) at 220 nM=100; Rt=1; LRMS m/z Calcd for C25 H33 N5 403.6; obsd LRMS APCI (M+1) m/z 404.0.

### EXAMPLE 66

### 5-[(R)-3-(2-Ethoxy-ethoxy)-pyrrolidin-1-ylmethyl]-2-pyrrolidin-1-ylmethyl-1H-indole

Reaction was conducted using the general procedure A; purification was accomplished using purification method A; isolated weight=8.8 mg; HPLC purity (%) at 220 nM=100; Rt=1.21; LRMS m/z Calcd for C22 H33 N3 O2 371.5; obsd LRMS APCI (M+1) m/z 372.0.

### EXAMPLE 67

### N,N-Dimethyl-2-[1-(2-pyrrolidin-1-ylmethyl-1H-indol-5-ylmethyl)-piperidin-4-yloxy]-acetamide

Reaction was conducted using the general procedure A; purification was accomplished using purification method A; isolated weight=8.7 mg; HPLC purity (%) at 220 nM=100; Rt=1.1; LRMS m/z Calcd for C23 H34 N4 O2 398.5; obsd LRMS APCI (M+1) m/z 399.0.

### EXAMPLE 68

### 2-Methyl-1-{4-[(2-pyrrolidin-1-ylmethyl-1H-indol-5-ylmethyl)-amino]-piperidin-1-yl}-propan-1-one

Reaction was conducted using the general procedure A; purification was accomplished using purification method A; isolated weight=9.3 mg; HPLC purity (%) at 220 nM=100; Rt=1.21; LRMS m/z Calcd for C23 H34 N4 O 382.5; obsd LRMS APCI (M+1) m/z 383.0.

### EXAMPLE 69

### 5-[(R3-(3-Methoxy-propoxy)-pyrrolidin-1-ylmethyl]-2-pyrrolidin-1-ylmethyl-1H-indole

Reaction was conducted using the general procedure A; purification was accomplished using purification method A; isolated weight=8.8 mg; HPLC purity (%) at 220 nM=90; Rt=1.21; LRMS m/z Calcd for C22 H33 N3 O2 371.5; obsd LRMS APCI (M+1) m/z 372.0.

### EXAMPLE 70

### General Procedure A

### [2-(4-Chloro-phenoxy)-5-fluoro-benzyl]-methyl-(2-pyrrolidin-1-ylmethyl-1H-indol-5-ylmethyl)-amine

Reaction was conducted using the general procedure A; purification was accomplished using purification method A; isolated weight=5.9 mg; HPLC purity (%) at 220 nM=100; Rt=1.77; LRMS m/z Calcd for C28 H29 LRMS m/z Calcd for Cl F N3 O 478.0; obsd LRMS APCI (M+1) m/z 478.0.

### EXAMPLE 71

### N,N-Dimethyl-2-[(R)-1-(2-pyrrolidin-1-ylmethyl-1H-indol-5-ylmethyl)-pyrrolidin-3-yloxy]-acetamide

Reaction was conducted using the general procedure A; purification was accomplished using purification method A; isolated weight=10 mg; HPLC purity (%) at 220 nM=100; Rt=1.1; LRMS m/z Calcd for C22 H32 N4 O2 384.5; obsd LRMS APCI (M+1) m/z 385.0.

### EXAMPLE 72

### N-tert-Butyl-2-[4-(2-pyrrolidin-1-ylmethyl-1H-indol-5-ylmethyl)-piperazin-1-yl]-nicotinamide

Reaction was conducted using the general procedure A; purification was accomplished using purification method A; isolated weight=16 mg; HPLC purity (%) at 220 nM=90; Rt=1.35; LRMS m/z Calcd for C28 H38 N6 O 474.6; obsd LRMS APCI (M+1) m/z 475.0.

### EXAMPLE 73

### 5-[(R)-2-(3-Ethyl-[1,2,4]oxadiazol-5-yl)-pyrrolidin-1-ylmethyl]-2-pyrrolidin-1-ylmethyl-1H-indole

Reaction was conducted using the general procedure A; purification was accomplished using purification method A; isolated weight=8.9 mg; HPLC purity (%) at 220 nM=100; Rt=1.18; LRMS m/z Calcd for C22 H29 N5 O 379.5; obsd LRMS APCI (M+1) m/z 380.0.

### EXAMPLE 74

### 5-[(R)-2-(3-Methyl-[1,2,4]oxadiazol-5-yl)-pyrrolidin-1-ylmethyl]-2-pyrrolidin-1-ylmethyl-1H-indole

Reaction was conducted using the general procedure A; purification was accomplished using purification method A; isolated weight=9.3 mg; HPLC purity (%) at 220 nM=100; Rt=1.1; LRMS m/z Calcd for C21 H27 N5 O 365.5; obsd LRMS APCI (M+1) m/z 366.0.

### EXAMPLE 75

### 5-[(S)-3-(2-Methoxy-ethoxy)-pyrrolidin-1-ylmethyl]-2-pyrrolidin-1-ylmethyl-1H-indole

Reaction was conducted using the general procedure A; purification was accomplished using purification method A; isolated weight=8.4 mg; HPLC purity (%) at 220 nM=100; Rt=1.14; LRMS m/z Calcd for C21 H31 N3 O2 357.5; obsd LRMS APCI (M+1) m/z 358.0.

### EXAMPLE 76

### Ethyl-pyridin-3-ylmethyl-(2-pyrrolidin-1-ylmethyl-1H-indol-5-ylmethyl)-amine

Reaction was conducted using the general procedure A; purification was accomplished using purification method A; isolated weight=2.5 mg; HPLC purity (%) at 220 nM=100; Rt=1.31; LRMS m/z Calcd for C22 H28 N4 348.5; obsd LRMS APCI (M+1) m/z 349.0.

### EXAMPLE 77

### 5-[(S)-2-(3-Ethyl-[1,2,4]oxadiazol-5-yl)-pyrrolidin-1-ylmethyl]-2-pyrrolidin-1-ylmethyl-1H-indole

Reaction was conducted using the general procedure A; purification was accomplished using purification method A; isolated weight=5.3 mg; HPLC purity (%) at 220 nM=100; Rt=1.18; LRMS m/z Calcd for C22 H29 N5 O 379.5; obsd LRMS APCI (M+1) m/z 380.0.

### EXAMPLE 78

### 5-[(S)-2-(3-Methyl-[1,2,4]oxadiazol-5-yl)-pyrrolidin-1-ylmethyl]-2-pyrrolidin-1-ylmethyl-1H-indole

Reaction was conducted using the general procedure A; purification was accomplished using purification method A; isolated weight=7.8 mg; HPLC purity (%) at 220 nM=100; Rt=1.1; LRMS m/z Calcd for C21 H27 N5 O 365.5; obsd LRMS APCI (M+1) m/z 366.0.

### EXAMPLE 80

### 5-[(S)-3-(3-Methoxy-propoxy)-pyrrolidin-1-ylmethyl]-2-pyrrolidin-1-ylmethyl-1H-indole

Reaction was conducted using the general procedure A; purification was accomplished using purification method A; isolated weight=4.9 mg; HPLC purity (%) at 220 nM=100; Rt=1.21; LRMS m/z Calcd for C22 H33 N3 O2 371.5; obsd LRMS APCI (M+1) m/z 372.0.

### EXAMPLE 81

### 1-{4-[(2-pyrrolidin-1-ylmethyl-1H-indol-5-ylmethyl)-amino]-piperidin-1-yl}-propan-1-one

Reaction was conducted using the general procedure A; purification was accomplished using purification method A; isolated weight=6.1 mg; HPLC purity (%) at 220 nM=100; Rt=1.14; LRMS m/z Calcd for C22 H32 N4 O 368.5; obsd LRMS APCI (M+1) m/z 369.0.

### EXAMPLE 82

### 5-[4-(3-Methyl-pyridin-2-yl)-[1,4]diazepan-1-ylmethyl]-2-pyrrolidin-1-ylmethyl-1H-indole

Reaction was conducted using the general procedure A; purification was accomplished using purification method A; isolated weight=10.1 mg; HPLC purity (%) at 220 nM=100; Rt=1.06; LRMS m/z Calcd for C25 H33 N5 403.6; obsd LRMS APCI (M+1) m/z 404.0.

### Determination of Biological Activity

The in vitro affinity of the compounds in the present invention at the rat or human histamine H3 receptors can be determined according to the following procedure. Frozen rat frontal brain or frozen human post-mortem frontal brain is homogenized in 20 volumes of cold 50 mM Tris HCl containing 2 mM MgCl₂ (pH to 7.4 at 4 degrees C.). The homogenate is then centrifuged at 45,000 G for 10 minutes. The supernatant is decanted and the membrane pellet re-suspended by Polytron in cold 50 mM Tris HCl containing 2 mM MgCl₂ (pH to 7.4 at 4 degrees C.) and centrifuged again. The final pellet is re-suspended in 50 mM Tris HCl containing 2 mM MgCl₂ (pH to 7.4 at 25 degrees C.) at a concentration of 12 mg/mL. Dilutions of compounds are made in 10% DMSO/50 mM Tris buffer (pH 7.4) (at 10× final concentration, so that the final DMSO concentration is 1%). Incubations are initiated by the addition of membranes (200 microliters) to 96 well V-bottom polypropylene plates containing 25 microliters of drug dilutions and 25 microliters of radioligand (1 nM final concentration ³H-N-methylhistamine). After a 1 hour incubation, assay samples are rapidly filtered through Whatman GF/B filters and rinsed with ice-cold 50 mM Tris buffer (pH 7.4) using a Skatron cell harvester. Radioactivity is quantified using a BetaPlate scintillation counter. The percent inhibition of specific binding can then be determined for each dose of the compound, and an IC50 or Ki value can be calculated from these results.

1. A compound of formula I 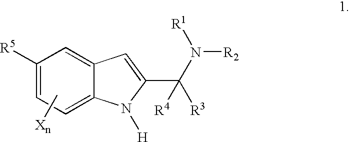
or a pharmaceutically acceptable salt thereof, wherein:
n=1, 2, or 3
Xₙ are independently selected from H, F, Cl, Br, I, C₁-C₆ alkyl (optionally substituted by F), C₁-C₆ alkoxyl (optionally substituted by F), (C₁-C₆ alkyl)-S(O)ₚ (optionally substituted by F, NO₂, COOH, COOR⁹, CONR¹⁰R¹¹;
wherein R⁹ is hydrogen, C₁-C₆ alkyl (optionally substituted by F), aryl, heteroaryl, C₁-C₆ alkyl-aryl, C₁-C₆ alkyl-heteroaryl;
R¹⁰ and R¹¹ are chosen from the group consisting of hydrogen, C₁-C₆ alkyl, aryl, heteroaryl, C₁-C₆ alkyl-(aryl), or R¹⁰ and R¹¹ taken together with the nitrogen to which they are attached form a ring of 4-8 atoms with up to 3 additional heteroatoms including N, O, S; and
p=0, 1 or 2.
R¹ and R² are independently selected from the group consisting of
hydrogen;
C₁-C₈ alkyl optionally substituted with 1 to 4 halogens or OH;
C₃-C₇ cycloalkyl;
3-8-membered heterocycloalkyl optionally substituted with a C₁-C₄ alkyl-carbonyl group;
C₆-C₁₀ arylsulfonyl optionally substituted with C₁-C₂ alkyl; and
5-10-membered heteroaryl;
R³ is selected from the group consisting of
C₁-C₈ alkyl optionally substituted with 1 to 4 halogens;
C₃-C₇ cycloalkyl;
C₆-C₁₄ aryl; or
R¹ and R² together with the nitrogen of the NR¹R² group form a 4-7 member ring, wherein one of the carbons in the ring is optionally replaced by O, S, NR⁶, or CO, and the ring is optionally fused to a C₆-C₁₀ arylene and is optionally substituted at a ring carbon with one or two C₁-C₄ alkyl groups, wherein R⁶ is
hydrogen;
C₁-C₈ alkyl optionally substituted with 1 to 4 halogens;
5-10-membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, cyano;
C₆-C₁₀ aryl optionally substituted with one or two C₁-C₂ alkyl; or
C₁-C₄ alkyl-carbonyl; or
R¹ and R³ together with the nitrogen of the NR¹R³ group form a 4-7 member ring, wherein one of the carbons in the ring is optionally replaced by O, S, NR^{6′}, or CO, and the ring is optionally fused to a C₆-C₁₀ arylene and is optionally substituted at a ring carbon with one or two C₁-C₄ alkyl groups, wherein R^{6′} is
hydrogen;
C₁-C₈ alkyl optionally substituted with 1 to 4 halogens;
5-10-membered heteroaryl optionally substituted with a substituent selected from the group consisting of halogen, C₁-C₄ alkyl, C₁-C₂ alkoxy, C₆-C₁₀ aryl, C₁-C₄ alkylaminocarbonyl, cyano;
C₆-C₁₀ aryl optionally substituted with one or two C₁-C₂ alkyl; or
C₁-C₄ alkyl-carbonyl;
R⁴ is
hydrogen, or
C₁-C₈ alkyl optionally substituted with 1 to 4 halogens;
R⁵ is —CHR^{7′}NR^{2″}R³;
R^{2″} is hydrogen, C₁-C₈ alkyl optionally substituted with 1 to 4 halogens, C₃-C₇ cycloalkyl-C₀-C₄ alkyl, C₆-C₁₄ aryl-C₀-C₄ alkyl, 5-10-membered heteroaryl-C₀-C₄ alkyl, or C₆-C₁₄ aryl-C₀-C₄ alkylene-O—C₀-C₄ alkyl, wherein each C₀-C₄ alkyl and each C₀-C₄ alkylene is optionally substituted with one to four C₁-C₄ alkyl;
R^{3″} is hydrogen, C₁-C₈ alkyl optionally substituted with 1 to 4 halogens, C₆-C₁₄ aryl, C₆-C₁₄ arylcarbonyl-C₆-C₁₄ aryl, C₆-C₁₄ arylcarbonyl-3-8-membered heterocycloalkyl, C₃-C₈ cycloalkylcarbonyl-C₆-C₁₄ aryl, C₃-C₈ cycloalkylcarbonyl-3-8-membered heterocycloalkyl, 3-8-membered heterocycloalkyl, 3-8-membered heterocycloalkylcarbonyl-C₆-C₁₄ aryl, or 3-8-membered heterocycloalkylcarbonyl-3-8-membered heterocycloalkyl;
or R^{3″} and R^{2″} together with the nitrogen of the CHR^{7′}NR^{2″}R^{3″} group form a second 5-, 6- or 7-membered aliphatic ring, wherein one of the carbons in the second 5-, 6-, or 7-membered aliphatic ring is optionally replaced by O, S, NR¹¹, or C═O and the second 5-, 6-, or 7-membered aliphatic ring is optionally substituted with one or two C₁-C₄ alkyl, wherein R¹¹ is hydrogen, C₁-C₈ alkyl optionally substituted with 1 to 4 halogens, C₃-C₇ cycloalkyl-C₀-C₄ alkyl, C₆-C₁₄ aryl-C₀-C₄ alkyl, 5-10-membered heteroaryl-C₀-C₄ alkyl, or C₆-C₁₄ aryl-C₀-C₄ alkylene-O—C₀-C₄ alkyl, wherein each C₀-C₄ alkyl and each C₀-C₄ alkylene is optionally substituted with one to four C₁-C₄ alkyl; and
R^{7′} is hydrogen, C₁-C₈ alkyl optionally substituted with 1 to 4 halogens, C₃-C₇ cycloalkyl-C₀-C₄ alkyl, C₆-C₁₄ aryl-C₀-C₄ alkyl, 5-10-membered heteroaryl-C₀-C₄ alkyl, C₆-C₁₄ aryl-C₀-C₄ alkylene-O—C₀-C₄ alkyl, wherein each C₀-C₄ alkyl and each C₀-C₄ alkylene is optionally substituted with one to four C₁-C₄ alkyl, or SO₂C₁-C₁₀ alkyl.

2. The compound of claim 1 wherein R¹ and R² together with the nitrogen to which they are attached form the 5-membered pyrrolidine ring.

3. The compound of claim 1 wherein R¹ and R³ together with the nitrogen to which they are attached form a 5-membered pyrrolidine ring.

4. The compound of claim 1 wherein R¹ and R² together with the nitrogen to which they are attached form the 6-membered piperidine ring.

5. The compound of claim 1 wherein R¹ and R³ together with the nitrogen to which they are attached form the 6-membered piperidine ring.

6. The compound of claim 1 wherein R¹ and R² together with the nitrogen to which they are attached form the 5-membered pyrrolidine ring, and R^{2″} and R^{3″} together with the nitrogen to which they are attached form the 6-membered substituted piperizine or morpholine ring.

7. The compound of claim 1, wherein R¹ is methyl, R² is methyl.

8. The compounds of formula I in of claim 1 wherein the compound is selected from the group consisting of:
2,5-Bis-pyrrolidin-1-ylmethyl-1H-indole.
5-Morpholin-4-ylmethyl-2-pyrrolidin-1-ylmethyl-1H-indole.
2-Pyrrolidin-1-ylmethyl-5-thiomorpholin-4-ylmethyl-1H-indole.
1-[4-(2-Pyrrolidin-1-ylmethyl-1H-indol-5-ylmethyl)-piperazin-1-yl]-ethanone.
6-[4-(2-Pyrrolidin-1-ylmethyl-1H-indol-5-ylmethyl)-piperazin-1-yl]-nicotinonitrile.
2-[Ethyl-(2-pyrrolidin-1-ylmethyl-1H-indol-5-ylmethyl)-amino]-ethanol.
1-(2-Pyrrolidin-1-ylmethyl-1H-indol-5-ylmethyl)-piperidin-4-ol.
1-[4-Phenyl-1-(2-pyrrolidin-1-ylmethyl-1H-indol-5-ylmethyl)-piperidin-4-yl]-ethanone.
Isopropyl-(2-pyrrolidin-1-ylmethyl-1H-indol-5-ylmethyl)-(1,3,5-trimethyl-1H-pyrazol-4-ylmethyl)-amine.
N-Butyl-N-methyl-N′-(2-pyrrolidin-1-ylmethyl-1H-indol-5-ylmethyl)-ethane-1,2-diamine.
5-(4-Pyrimidin-2-yl-[1,4]diazepan-1-ylmethyl)-2-pyrrolidin-1-ylmethyl-1H-indole.
Cyclopropyl-(2-pyrrolidin-1-ylmethyl-1H-indol-5-ylmethyl)-(1,3,5-trimethyl-1H-pyrazol-4-ylmethyl)-amine.
5-[4-(1-Methyl-1H-imidazol-2-ylmethyl)piperazin-1-ylmethyl]-2-pyrrolidin-1-ylmethyl-1H-indole.
[4-(2-Pyrrolidin-1-ylmethyl-1H-indol-5-ylmethyl)-piperazin-1-yl]-acetic acid methyl ester.
Methyl-(3-methyl-pyridin-2-ylmethyl)-(2-pyrrolidin-1-ylmethyl-1H-indol-5-ylmethyl)-amine.
(2-Furan-2-yl-ethyl)-(2-pyrrolidin-1-ylmethyl-1H-indol-5-ylmethyl)-amine.
5-[4-(2-Methyl-thiazol-4-ylmethyl)-piperazin-1-ylmethyl]-2-pyrrolidin-1-ylmethyl-1H-indole.
1-Phenyl-4-(2-pyrrolidin-1-ylmethyl-1H-indol-5-ylmethyl)-piperazin-2-one.
Methyl-(5-propyl-1H-pyrazol-3-ylmethyl)-(2-pyrrolidin-1-ylmethyl-1H-indol-5-ylmethyl)-amine.
2-Cyclopropyl-6-(2-pyrrolidin-1-ylmethyl-1H-indol-5-ylmethyl)-5,6,7,8-tetrahydro-pyrido[4,3-d]pyrimidine.
(4-Phenyl-thiazol-2-ylmethyl)-(2-pyrrolidin-1-ylmethyl-1H-indol-5-ylmethyl)-amine.
1-(3,5-Dimethyl-phenyl)-4-[(2-pyrrolidin-1-ylmethyl-1H-indol-5-yl)methyl]-piperazin-2-one.
(6-Methyl-imidazo[1,2-a]pyridin-2-ylmethyl)-(2-pyrrolidin-1-ylmethyl-1H-indol-5-ylmethyl)-amine.
(3-Pyrazol-1-yl-benzyl)-(2-pyrrolidin-1-ylmethyl-1H-indol-5-ylmethyl)-amine.
(1-Methyl-1H-imidazol-2-ylmethyl)-(2-pyrrolidin-1-ylmethyl-1H-indol-5-ylmethyl)-amine.
(2-Imidazo[1,2-a]pyridin-2-yl-ethyl)-(2-pyrrolidin-1-ylmethyl-1H-indol-5-ylmethyl)-amine.
(7-Methyl-imidazo[1,2-a]pyridin-2-ylmethyl)-(2-pyrrolidin-1-ylmethyl-1H-indol-5-ylmethyl)-amine.
1-(4-Fluoro-benzyl)-4-(2-pyrrolidin-1-ylmethyl-1H-indol-5-ylmethyl)-piperazin-2-one.
Methyl-(2-pyrrolidin-1-ylmethyl-1H-indol-5-ylmethyl)-quinoxalin-2-ylmethyl-amine.
5-(4-Morpholin-4-yl-piperidin-1-ylmethyl)-2-pyrrolidin-1-ylmethyl-1H-indole.
(5-Methyl-imidazo[1,2-a]pyridin-2-ylmethyl)-(2-pyrrolidin-1-ylmethyl-1H-indol-5-ylmethyl)-amine.
Imidazo[1,2-a]pyridin-2-ylmethyl-(2-pyrrolidin-1-ylmethyl-1H-indol-5-ylmethyl)-amine.
(2-Methoxy-2-methyl-propyl)-(2-pyrrolidin-1-ylmethyl-1H-indol-5-ylmethyl)-amine.
(1,5-Dimethyl-1H-pyrazol-4-ylmethyl)-(2-pyrrolidin-1-ylmethyl-1H-indol-5-ylmethyl)-amine.
[2-(3,5-Dimethyl-pyrazol-1-yl)-ethyl]-methyl-(2-pyrrolidin-1-ylmethyl-1H-indol-5-ylmethyl)-amine.
6-(2-Pyrrolidin-1-ylmethyl-1H-indol-5-ylmethyl)-2-trifluoromethyl-5,6,7,8-tetrahydro-pyrido[4,3-d]pyrimidine.
Methyl-(4-methyl-1H-imidazol-2-ylmethyl)-(2-pyrrolidin-1-ylmethyl-1H-indol-5-ylmethyl)-amine.
Methyl-[2-(4-methyl-thiazol-5-yl)-ethyl]-(2-pyrrolidin-1-ylmethyl-1H-indol-5-ylmethyl)-amine.
[2-(3,5-Dimethyl-pyrazol-1-yl)-ethyl]-(2-pyrrolidin-1-ylmethyl-1H-indol-5-ylmethyl)-amine.
2-Pyridin-2-yl-6-(2-pyrrolidin-1-ylmethyl-1H-indol-5-ylmethyl)-5,6,7,8-tetrahydro-pyrido[4,3-d]pyrimidine.
Methyl-(2-pyrrolidin-1-ylmethyl-1H-indol-5-ylmethyl)-(tetrahydro-pyran-4-ylmethyl)-amine.
2-Methoxy-6-(2-pyrrolidin-1-ylmethyl-1H-indol-5-ylmethyl)-5,6,7,8-tetrahydro-pyrido[4,3-d]pyrimidine.
Methyl-pyrimidin-4-ylmethyl-(2-pyrrolidin-1-ylmethyl-1H-indol-5-ylmethyl)-amine
2-Pyridin-4-yl-6-(2-pyrrolidin-1-ylmethyl-1H-indol-5-ylmethyl)-5,6,7,8-tetrahydro-pyrido[4,3-d]pyrimidine.
(5-Phenyl-isoxazol-3-ylmethyl)-(2-pyrrolidin-1-ylmethyl-1H-indol-5-ylmethyl)-amine.
Benzothiazol-2-ylmethyl-(2-pyrrolidin-1-ylmethyl-1H-indol-5-ylmethyl)-amine.
(4-Methyl-thiazol-2-ylmethyl)-(2-pyrrolidin-1-ylmethyl-1H-indol-5-ylmethyl)-amine.
(3-Phenyl-[1,2,4]oxadiazol-5-ylmethyl)-(2-pyrrolidin-1-ylmethyl-1H-indol-5-ylmethyl )-amine.
Methyl-(3-phenyl-[1,2,4]oxadiazol-5-ylmethyl)-(2-pyrrolidin-1-ylmethyl-1H-indol-5-ylmethyl)-amine.
4-(2-Pyrrolidin-1-ylmethyl-1H-indol-5-ylmethyl)-2 ,3,4,5-tetrahydro-benzo[f][1,4]oxazepine.
2-Pyrrolidin-1-ylmethyl-5-(4-thiophen-3-ylmethyl-piperazin-1-ylmethyl)-1H-indole.
Methyl-(5-phenyl-[1,3,4]oxadiazol-2-ylmethyl)-(2-pyrrolidin-1-ylmethyl-1H-indol-5-ylmethyl)-amine.
Methyl-(4-phenyl-thiazol-2-ylmethyl)-(2-pyrrolidin-1-ylmethyl-1H-indol-5-ylmethyl)-amine.
Methyl-(5-methyl-1H-pyrazol-3-ylmethyl)-(2-pyrrolidin-1-ylmethyl-1H-indol-5-ylmethyl)-amine.
(2-Pyrrolidin-1-ylmethyl-1H-indol-5-ylmethyl)-(4,5,6,7-tetrahydro-benzothiazol-2-ylmethyl)-amine.
5-((R)-2-Morpholin-4-ylmethyl-pyrrolidin-1-ylmethyl)-2-pyrrolidin-1-ylmethyl-1H-indole.
5-((S)-3-Morpholin-4-yl-pyrrolidin-1-ylmethyl)-2-pyrrolidin-1-ylmethyl-1H-indole.
5-[(S)-2-(4-Methyl-piperazin-1-ylmethyl)-pyrrolidin-1-ylmethyl]-2-pyrrolidin-1-ylmethyl-1H-indole.
(2-Phenyl-oxazol-4-ylmethyl)-(2-pyrrolidin-1-ylmethyl-1H-indol-5-ylmethyl)-amine.
5-[(S)-3-(2-Ethoxy-ethoxy)-pyrrolidin-1-ylmethyl]-2-pyrrolidin-1-ylmethyl-1H-indole.
2-[4-(2-Pyrrolidin-1-ylmethyl-1H-indol-5-ylmethyl)-piperazin-1-yl]-nicotinamide.
(R)-4-Methanesulfonyl-1-(2-pyrrolidin-1-ylmethyl-1H-indol-5-ylmethyl)-piperazine-2-carboxylic acid methyl ester.
5-(3-Morpholin-4-yl-azetidin-1-ylmethyl)-2-pyrrolidin-1-ylmethyl-1H-indole.
5-((S)-3-Propoxy-pyrrolidin-1-ylmethyl)-2-pyrrolidin-1-ylmethyl-1H-indole.
5-[4-(6-Methyl-pyridin-2-yl)-[1,4]diazepan-1-ylmethyl]-2-pyrrolidin-1-ylmethyl-1H-indole.
5-[(R)-3-(2-Ethoxy-ethoxy)-pyrrolidin-1-ylmethyl]-2-pyrrolidin-1-ylmethyl-1H-indole.
N,N-Dimethyl-2-[1-(2-pyrrolidin-1-ylmethyl-1H-indol-5-ylmethyl)-piperidin-4-yloxy]-acetamide.
2-Methyl-1-{4-[(2-pyrrolidin-1-ylmethyl-1H-indol-5-ylmethyl)-amino]-piperidin-1-yl}-propan-1-one.
5-[(R)-3-(3-Methoxy-propoxy)-pyrrolidin-1-ylmethyl]-2-pyrrolidin-1-ylmethyl-1H-indole.
[2-(4-Chloro-phenoxy)-5-fluoro-benzyl]-methyl-(2-pyrrolidin-1-ylmethyl-1H-indol-5-ylmethyl)-amine.
N,N-Dimethyl-2-[(R)-1-(2-pyrrolidin-1-ylmethyl-1H-indol-5-ylmethyl)-pyrrolidin-3-yloxy]-acetamide.
N-tert-Butyl-2-[4-(2-pyrrolidin-1-ylmethyl-1H-indol-5-ylmethyl)-piperazin-1-yl]-nicotinamide.
5-[(R)-2-(3-Ethyl-[1,2,4]oxadiazol-5-yl)-pyrrolidin-1-ylmethyl]-2-pyrrolidin-1-ylmethyl-1H-indole.
5-[(R)-2-(3-Methyl-[1,2,4]oxadiazol-5-yl)-pyrrolidin-1-ylmethyl]-2-pyrrolidin-1-ylmethyl-1H-indole.
5-[(S)-3-(2-Methoxy-ethoxy)-pyrrolidin-1-ylmethyl]-2-pyrrolidin-1-ylmethyl-1H-indole.
Ethyl-pyridin-3-ylmethyl-(2-pyrrolidin-1-ylmethyl-1H-indol-5-ylmethyl)-amine.
5-[(S)-2-(3-Ethyl-[1,2,4]oxadiazol-5-yl)-pyrrolidin-1-ylmethyl]-2-pyrrolidin-1-ylmethyl-1H-indole.
5-[(S)-2-(3-Methyl-[1,2,4]oxadiazol-5-yl)-pyrrolidin-1-ylmethyl]-2-pyrrolidin-1-ylmethyl-1H-indole.
5-[(S)-3-(3-Methoxy-propoxy)-pyrrolidin-1-ylmethyl]-2-pyrrolidin-1-ylmethyl-1H-indole.
1-{4-[(2-Pyrrolidin-1-ylmethyl-1H-indol-5-ylmethyl)-amino]-piperidin-1-yl}propan-1-one.
5-[4-(3-Methyl-pyridin-2-yl)-[1,4]diazepan-1-ylmethyl]-2-pyrrolidin-1-ylmethyl-1H-indole.

9. The compounds:
3-Iodo-4-(2,2,2-trifluoro-acetylamino)-benzoic acid methyl ester;
2-Hydroxymethyl-1H-indole-5-carboxylic acid methyl ester; and
2-Formyl-1H-indole-5-carboxylic acid methyl ester.

10. The compounds:
2-Pyrrolidin-1-ylmethyl-1H-indole-5-carboxylic acid methyl ester;
(2-Pyrrolidin-1-ylmethyl-1H-indol-5-yl)-methanol; and
2-Pyrrolidin-1-ylmethyl-1H-indole-5-carbaldehyde.

11. A pharmaceutical composition for treating a disorder or condition that may be treated by antagonizing histamine-3 receptors, the composition comprising a compound of formula I as described in claim 1, and optionally a pharmaceutically acceptable carrier.

12. A method of treatment of a disorder or condition that may be treated by antagonizing histamine-3 receptors, the method comprising administering to a mammal in need of such treatment a compound of formula I as described in claim 1.

13. A pharmaceutical composition comprising a compound of formula I as described in claim 1, and optionally a pharmaceutically acceptable carrier.

14. A method of treatment of a disorder or condition selected from the group consisting of depression, mood disorders, schizophrenia, anxiety disorders, Alzheimer's disease, attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro- intestinal tract, the method comprising administering to a mammal in need of such treatment a compound of formula I as described in claim 1.

15. The method of claim 14, wherein the disorder or condition is selected from the group consisting of anxiety disorders, attention-deficit hyperactivity disorder, respiratory diseases, and obesity.

16. The method of claim 14, wherein the disorder or condition is a respiratory disease selected from the group consisting of adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

17. A pharmaceutical composition for treating allergic rhinitis, nasal congestion or allergic congestion comprising
(a) an H3 receptor antagonist compound of formula 1; or a pharmaceutically acceptable salt thereof;
(b) an H1 receptor antagonist or a pharmaceutically acceptable salt thereof; and
(c) a pharmaceutically acceptable carrier;
wherein the active ingredients (a) and (b) above are present in amounts that render the composition effective in treating allergy rhinitis, nasal congestion or allergic congestion

18. A pharmaceutical composition for treating depression and mood disorder comprising:
a) an H3 receptor antagonist compound of Formula 1 or a pharmaceutically acceptable salt thereof;
b) a neurotransmitter re-uptake blocker or a pharmaceutically acceptable salt thereof;
c) a pharmaceutically acceptable carrier;
wherein the active ingredients (a) and (b) above are present in amounts that render the composition effective in treating depression and mood disorder.

19. The composition according to claim 18 wherein the H3 receptor antagonist and the neurotransmitter blocker are given simultaneously.

20. The composition according to claim 17 wherein the H3 receptor antagonist and the H1 receptor antagonist are given simultaneously.

21. The pharmaceutical composition of claim 18 wherein the neurotransmitter uptake blocker are selected the group consisting of sertraline, fluoxetine and paroxetine.

22. the pharmaceutical composition of claim 17, wherein the H1 receptor antagonist is certirizine.
